SEQUENCE LISTING (54 sequences)

SEQ ID NO: 1 - DNA, 20 bp, Artificial Sequence
  Description of Artificial Sequence Synthetic primer
aaagcttggcgcagatgtga

SEQ ID NO: 2 - DNA, 20 bp, Artificial Sequence
  Description of Artificial Sequence Synthetic primer
ttgccacggcattggtaaga

SEQ ID NO: 3 - DNA, 20 bp, Artificial Sequence
  Description of Artificial Sequence Synthetic primer
tttaccaactgccagccaga

SEQ ID NO: 4 - DNA, 20 bp, Artificial Sequence
  Description of Artificial Sequence Synthetic primer
tgttgctgcagtcacaccat

SEQ ID NO: 5 - DNA, 22 bp, Artificial Sequence
  Description of Artificial Sequence Synthetic primer
gagaagctgttccagatagtgc

SEQ ID NO: 6 - DNA, 22 bp, Artificial Sequence
  Description of Artificial Sequence Synthetic primer
ctcagggtacatgaaatggtgg

SEQ ID NO: 7 - DNA, 22 bp, Artificial Sequence
  Description of Artificial Sequence Synthetic primer
atgtacaacatgatggagacgg

SEQ ID NO: 8 - DNA, 20 bp, Artificial Sequence
  Description of Artificial Sequence Synthetic primer
ccacaccatgaaggcattca

SEQ ID NO: 9 - DNA, 20 bp, Artificial Sequence
  Description of Artificial Sequence Synthetic primer
tttgccaagctcctgaagca

SEQ ID NO: 10 - DNA, 20 bp, Artificial Sequence
  Description of Artificial Sequence Synthetic primer
aaagcggcagatggtcgttt

SEQ ID NO: 11 - DNA, 24 bp, Artificial Sequence
  Description of Artificial Sequence Synthetic primer
gagcatgcagaagcgcagatcaaa

SEQ ID NO: 12 - DNA, 24 bp, Artificial Sequence
  Description of Artificial Sequence Synthetic primer
tatggctgatgctctggcagaagt

SEQ ID NO: 13 - DNA, 22 bp, Artificial Sequence
  Description of Artificial Sequence Synthetic primer
tcagagacagaaatacctcagc

SEQ ID NO: 14 - DNA, 20 bp, Artificial Sequence
  Description of Artificial Sequence Synthetic primer
aggaagagtaaaggctgggg

SEQ ID NO: 15 - DNA, 20 bp, Artificial Sequence
  Description of Artificial Sequence Synthetic primer
ttcaacctggcggacatcaa

SEQ ID NO: 16 - DNA, 20 bp, Artificial Sequence
  Description of Artificial Sequence Synthetic primer
ttcagcacgaacttgcccat

SEQ ID NO: 17 - DNA, 20 bp, Artificial Sequence
  Description of Artificial Sequence Synthetic primer
gtattgccctttggaggcac

SEQ ID NO: 18 - DNA, 20 bp, Artificial Sequence
  Description of Artificial Sequence Synthetic primer
agggtcataactgggcactc

SEQ ID NO: 19 - DNA, 20 bp, Artificial Sequence
  Description of Artificial Sequence Synthetic primer
ttccagaacacgatgaggca

SEQ ID NO: 20 - DNA, 20 bp, Artificial Sequence
  Description of Artificial Sequence Synthetic primer
acctgtgacagcttggtaga

SEQ ID NO: 21 - DNA, 22 bp, Artificial Sequence
  Description of Artificial Sequence Synthetic primer
aaggtgtctagtaagacagcag

SEQ ID NO: 22 - DNA, 20 bp, Artificial Sequence
  Description of Artificial Sequence Synthetic primer
atcattttgcctagcccacc

SEQ ID NO: 23 - DNA, 20 bp, Artificial Sequence
  Description of Artificial Sequence Synthetic primer
atgcattctggcgaccatca

SEQ ID NO: 24 - DNA, 20 bp, Artificial Sequence
  Description of Artificial Sequence Synthetic primer
acgccattcccagtaccttg

SEQ ID NO: 25 - DNA, 20 bp, Artificial Sequence
  Description of Artificial Sequence Synthetic primer
agtggattgctccttgacga

SEQ ID NO: 26 - DNA, 20 bp, Artificial Sequence
  Description of Artificial Sequence Synthetic primer
gggcacgacctcatcttcta

SEQ ID NO: 27 - DNA, 21 bp, Artificial Sequence
  Description of Artificial Sequence Synthetic primer
accctagagccgaaaagaaag

SEQ ID NO: 28 - DNA, 20 bp, Artificial Sequence
  Description of Artificial Sequence Synthetic primer
gccgcacagtaatggtaagg

SEQ ID NO: 29 - DNA, 20 bp, Artificial Sequence
  Description of Artificial Sequence Synthetic primer
aagatctccgcctcgagaaa

SEQ ID NO: 30 - DNA, 20 bp, Artificial Sequence
  Description of Artificial Sequence Synthetic primer
gcagagatcctcactctccg

SEQ ID NO: 31 - DNA, 20 bp, Artificial Sequence
  Description of Artificial Sequence Synthetic primer
ctttgatgagagacgtcggg

SEQ ID NO: 32 - DNA, 20 bp, Artificial Sequence
  Description of Artificial Sequence Synthetic primer
cttcccacttttccgctctg

SEQ ID NO: 33 - DNA, 20 bp, Artificial Sequence
  Description of Artificial Sequence Synthetic primer
ggggacagtggtaaaagcaa

SEQ ID NO: 34 - DNA, 20 bp, Artificial Sequence
  Description of Artificial Sequence Synthetic primer
tccctgcgttccactatctt

SEQ ID NO: 35 - DNA, 21 bp, Artificial Sequence
  Description of Artificial Sequence Synthetic primer
gagttcaaagaagccttcagc

SEQ ID NO: 36 - DNA, 20 bp, Artificial Sequence
  Description of Artificial Sequence Synthetic primer
atccttgttcaccacccctt

SEQ ID NO: 37 - DNA, 20 bp, Artificial Sequence
  Description of Artificial Sequence Synthetic primer
ggtgctgaaggctgattacg

SEQ ID NO: 38 - DNA, 20 bp, Artificial Sequence
  Description of Artificial Sequence Synthetic primer
ttggaacatggcctctggat

SEQ ID NO: 39 - DNA, 20 bp, Artificial Sequence
  Description of Artificial Sequence Synthetic primer
ctgtgggctgttcaccaact

SEQ ID NO: 40 - DNA, 19 bp, Artificial Sequence
  Description of Artificial Sequence Synthetic primer
gccgcaaccaacacatagg

SEQ ID NO: 41 - DNA, 20 bp, Artificial Sequence
  Description of Artificial Sequence Synthetic primer
gggcagaatcatcacgaagt

SEQ ID NO: 42 - DNA, 20 bp, Artificial Sequence
  Description of Artificial Sequence Synthetic primer
tgttgtgctgtaggaagctc

SEQ ID NO: 43 - DNA, 20 bp, Artificial Sequence
  Description of Artificial Sequence Synthetic primer
gagcctgcgcaatggaataa

SEQ ID NO: 44 - DNA, 20 bp, Artificial Sequence
  Description of Artificial Sequence Synthetic primer
ataccctgtgggcttgttga

SEQ ID NO: 45 - DNA, 20 bp, Artificial Sequence
  Description of Artificial Sequence Synthetic primer
tcaacactccaaactgtgcc

SEQ ID NO: 46 - DNA, 21 bp, Artificial Sequence
  Description of Artificial Sequence Synthetic primer
agcaagtgaactgtggtccat

SEQ ID NO: 47 - DNA, 20 bp, Artificial Sequence
  Description of Artificial Sequence Synthetic primer
cgactggaagacacgtttgg

SEQ ID NO: 48 - DNA, 20 bp, Artificial Sequence
  Description of Artificial Sequence Synthetic primer
tttgggagtacggatgcact

SEQ ID NO: 49 - DNA, 21 bp, Artificial Sequence
  Description of Artificial Sequence Synthetic primer
cacaacagagccaacagagtc

SEQ ID NO: 50 - DNA, 20 bp, Artificial Sequence
  Description of Artificial Sequence Synthetic primer
tccaggtggcagaagtagac

SEQ ID NO: 51 - DNA, 20 bp, Artificial Sequence
  Description of Artificial Sequence Synthetic primer
agagtgagtccaactcggtg

SEQ ID NO: 52 - DNA, 20 bp, Artificial Sequence
  Description of Artificial Sequence Synthetic primer
agggcaaagtccaggatctc

SEQ ID NO: 53 - DNA, 23 bp, Artificial Sequence
  Description of Artificial Sequence Synthetic primer
ggagcatatgtgtcttcagctac

SEQ ID NO: 54 - DNA, 23 bp, Artificial Sequence
  Description of Artificial Sequence Synthetic primer
aagctggccattacgtagttttg

(19) United States
(12) Patent Application Publication
(10) Pub. No.: US 2020/0283726 A1
(43) Pub. Date: Sep. 10, 2020

(54) PROCESS FOR CONTINUOUS CELL CULTURE OF GPSCS

(71) Applicant: Georgetown University, Washington, DC (US)

(72) Inventors: Ian Gallicano, Alexandria, VA (US); Samiksha Mahapatra, Washington, DC (US); Dianna Martin, Woodbridge, VA (US)

(73) Assignee: Georgetown University, Washington, DC (US)

(21) Appl. No.: 16/759,352

(22) PCT Filed: Nov. 20, 2018
(86) PCT No.: PCT/US18/62009

§ 371 Date: Apr. 26, 2020

Related U.S. Application Data
(60) Provisional application No. 62/589,018, filed on Nov. 21, 2017.

(51) Int. Cl. C12N 5/0735

(52) U.S. Cl. CPC C12N 5/0611; C12Y 207/11001; C12N 2500/30

(57) ABSTRACT

The present invention is directed towards methods of culturing germline pluripotent stem cells (gPSCs), with the methods comprising culturing the cells in a cell culture medium while inhibiting the activity of Rho kinase (ROCK) in the cells during culture. The present invention is also directed towards methods of using these continuously cultured gPSCs.

### BACKGROUND OF THE INVENTION

### Field of the Invention

The present invention is directed towards methods of culturing germline pluripotent stem cells (gPSCs), with the methods comprising culturing the cells in a cell culture medium while inhibiting the activity of Rho kinase (ROCK) in the cells during culture. The present invention is also directed towards methods of using these continuously cultured gPSCs.

### Background of the Invention

It has previously been reported germline stem cells when removed from their niche have the ability to differentiate into cell types from all three germ layers (ecto, meso, and endoderm), thus these cells are often referred to a germline pluripotent stem cells (gPSCs).

When spermatogonial stem cells (SSCs) or ovarian stem cells (OSCs) are removed from their native environment, they can begin to express factors redefining their “stemness” from unipotent, i.e., only able to make sperm or eggs, respectively, to pluripotent. These redefined cells are known as germline pluripotent stem cells (gPSCs) and germline embryonic stem-like cells (gESLCs).

While gPSCs may hold promise for use in regenerative medicine, these cells grew very slowly. In fact, these cells grow slowly that it is, to date, not practical to utilize gPSCs in any type of regenerative medicine setting. Moreover, because so much time is required to expand these cells in culture, invariably large portions of the cells will begin to differentiate, rendering them unusable for further manipulation.

Accordingly, what is needed are methods of culturing gPSCs in a continuous manner that can promote rapid expansion, without differentiation.

### SUMMARY OF THE INVENTION

The present invention is directed towards methods of culturing germline pluripotent stem cells (gPSCs), with the methods comprising culturing the cells in a cell culture medium while inhibiting the activity of Rho kinase (ROCK) in the cells during culture. The present invention is also directed towards methods of using these continuously cultured gPSCs.

The present invention is also directed towards methods of producing conditionally immortalized gPSCs, with the methods comprising culturing the cells in the presence of a cell culture medium while inhibiting the activity of ROCK in the cells. Culturing the gPSCs in such conditions will produce conditionally immortalized gPSCs.

### BRIEF DESCRIPTION OF THE DRAWINGS

FIG. 1 depicts the structures of select ROCK inhibitors.

FIG. 2 depicts identification of SSCs within testes that gives rise to clonal gPSCs. FIG. 2A: Acting as a positive control, a single mouse E14 strain embryonic stem cell gives rise to a colony within 7 days of culture. FIG. 2B: After enzymatic digestion and filtration of human testes tissue, distinctive size differences among cells are clearly evident. “Small cells” (<5-7 μm in diameter), “medium cells” (˜8-12 μm in diameter), and “large cells” (>12-20 μm) cells were isolated using a mouth pipette and placed into a well of a 96 well plate. After 14 days of incubation wells were assessed for clonal growth. The medium size cells produced colonies through single cell colony expansion. FIGS. 2C-2F: Removing bFGF from the hESC medium induced de-differentiation at ˜21 days, as RT-PCR showed expression of genes from all three germ layers; alpha fetal protein (AFP-Endoderm), Bone morphogenic protein 4 (Bmp4-mesoderm), and nestin (ectoderm). GAPDH was the RT-PCR control gene. Surrounding fibroblasts did not show expression of these three specific genes. FIG. 2G: Shows quantifies of colony formation from the three sizes of cells. FIG. 2H show quantities of antibody expression of SSC markers, SSEA4 but not Gpr125 or Gfra1 identified the cells that produced colonies of gPSCs. SSEA4 positive cells are 8-12 μm in diameter (inset in FIG. 2B), while Gfra1 cells are much smaller (inset in FIG. 2B). The large cells were mostly vimentin positive (inset in FIG. 2B) most likely representing Sertoli cells.

FIG. 3 depicts cardiac cell lineages that can be produced from gPSCs. FIG. 3A: Cells of the SSC-enriched fraction are cultured in medium containing GDNF for four days. The fraction is full of cells positive for various SSC markers including SSEA4. FIG. 3B: After four days, the medium is switched to a basic hESC medium containing bFGF and serum replacement and incubated for at least 10 days, after which RT-PCR shows evidence of all four Yamanaka factors plus nanog and CD73. FIG. 3C: Switching hESC medium for cardiac differentiation medium results in growth and morphologically darker looking colonies. RT-PCR shows expression of 9 out of 10 cardiac genes within 10 days of differentiation. FIGS. 3D-3I: Confocal analyses show protein expression of specific cardiac genes including nuclear staining of Nkx2.5 (arrows). Arrows in FIGS. 3D and 3E point to areas positive for cardiac troponin (CnnT), while arrowheads point to nuclear Nkx2.5 staining. Dapi staining in FIG. 3E identifies the nuclei. Arrows in 3G and 3H point to distinct filaments of cardiac actin. The DIC image in 3H reveals the actin fibers within healthy cells. FIGS. 3J-3M: Transfection of colonies with a cMHC-GFP further confirms cardiac gene expression. Arrow in 3J points to a GFP positive colony. Arrowheads point to untransfected colonies. These untransfected colonies serve as an internal control ruling out autofluorescence. FIG. 3L: Arrows point to cells within the colony expressing cMHC-GFP. FIGS. 3K and 3M: Phase contrast views show healthy colonies.

FIG. 4 depicts differentiation of cardiac colonies beyond day 10 results in colonies that express pro-cardiac regenerative paracrine factors. FIG. 4A: RT-PCR shows expression of seven pro-cardiac regenerative paracrine factors. FIG. 4B-4D: Immunofluorescent and DIC analyses using antibodies directed against IGF-1 and NRG-1 show colonies staining positive for both paracrine factors. Arrowheads in 4B and 4C point to regions of variable staining within colonies. Asterisks highlight fibroblasts that can emanate from colonies, which show no fluorescent staining.

FIG. 5 depicts that culturing gPSC colonies in GEM allows for their rapid expansion without the loss of stemness. FIGS. 5A and 5B: Colonies of gPSC grown in hESC medium lose their colony structure beginning ˜5 days post switching to GEM. FIG. 5C: By day 10, most colonies become individual layers of cobble-stone shaped cells (outline shows region of cobblestone pattern of cells), which can be continuously cultured. FIG. 5D: Switching from GEM to hESC medium, colonies begin to re-form within 5 days. FIG. 5E: By day 10 after the switch to hESC medium gPSC colonies fully return.

FIGS. 5F and 5G: RT-PCR shows that these colonies express all the same stem cell factors prior to expansion, which is confirmed by confocal microscopy. Nuclear staining of Oct4, Nanog, Sox2, and Lin28 is prevalent. FIG. 5H: 21 days post differentiation, large dark colonies form, which are all positive for paracrine factor gene expression (FIG. 5I). FIG. 5J: Western analysis of the colonies show they are positive for the cardiac intermediate filament desmin similar to the mouse heart. Undifferentiated gPSCs are negative for desmin as are mouse embryonic fibroblasts (MEFs).

FIG. 6 depicts 500 gPSC colonies that were expanded by traditional conditions, i.e., by trypsinization and passaging 1:2, or were expanded 1:2 in GEM. Comparing two different patients, no marked difference was observed until the second and third passages where GEM-grown gPSCs grew ˜2× faster than conventional growth. By the fourth passage, GEM-grown colonies re-generated close to 4× more colonies when compared to conventional growth and passaging. More importantly, those ˜4× more colonies were obtained in almost half the time.

FIG. 7 depicts immunoprecipitation (IP) and Western analysis of culture medium that shows that paracrine factors are secreted from gPSC-derived cardiac colonies. FIG. 7A: Silver stained SDS-PAGE gel detected IGF-1 by IP after 12 hrs of culture, increasing in intensity through 48 hrs of culture. FIG. 7B: Silver staining shows TGFβ secretion within 12 hrs becoming more intense after 48 hrs. FIG. 7C: VEGF is detectable by about 24 hrs of culture. FIG. 7D: Western analysis of CTFG secretion is detected within 24 hrs while NRG-1 secretion (FIG. 7E-7F) is detected after 48 hrs of culture. Cardiac differentiation of gPSCs from two patients are shown for Nrg1. All IP experiments were run with a lane containing IgG alone to identify the heavy and light chain bands. 2 μl from all samples were analyzed using a nano-drop ND-8000 (Thermo Fisher Inc.) to normalize protein concentrations.

FIG. 8 depicts gPSC-derived cardiac colonies can fuse with beating cardiac tissue. FIG. 8A-8B: E9.5 fetal hearts were isolated from mouse embryos using Dumont #5 forceps. 10-15 fetal hearts were placed in one well of a 96 well plate and cMHC-GFP positive colonies were mouth pipetted into crevasses within the beating heart or simply overlaid onto the hearts. 24 hours later, hearts were analyzed live using a Leica stereoscope equipped with fluorescence. Hearts containing green areas were then fixed, stained for CNX43, and Dapi and visualized by confocal microscopy. FIG. 8C: Multiple GFP-positive regions were evident (arrows). FIG. 8D-8E: Higher magnification clearly showed GFP positive cells fused to cardiac tissue via gap junctions (arrowheads) on the same focal plane as surround heart tissue.

### DETAILED DESCRIPTION OF THE INVENTION

The present invention is directed towards methods of culturing germline pluripotent stem cells (gPSCs), with the methods comprising culturing the cells in a cell culture medium while inhibiting the activity of Rho kinase (ROCK) in the cells during culture. The present invention is also directed towards methods of using these continuously cultured gPSCs.

As used herein, the term “germline pluripotent stem cell” (gPSC) or “germline embryonic stem-like cells” (gESLCs) refers to a cell or cells that are derived from germline stem cells. As used herein, germline stem cells include spermatogonial stem cells (SSCs) and ovarian stem cells (OSCs). The term “spermatogonial stem cell” is well-known in the art and is used herein as it is in the art to mean a stem cell that is isolated from male sex organs, such as testes. Typically, SSCs are unipotent in that they can divide indefinitely in their native environment to produce another self-renewing stem cell and a daughter cell that can only differentiate into a sperm or male gamete or sex cell. Similarly, the term OSC is used herein as it is in the art to mean a stem cell that is isolated from female sex organs, such as ovaries. Again, OSCs are unipotent in that they can divide indefinitely in their native environment to produce another self-renewing stem cell and a daughter cell that can only differentiate into an egg or female gamete or sex cell. See Navaroli, D. M., et al., Methods Mol. Biol., 1457:253-268 (2016) and White, Y. A., et al., Nature Med., 18:413-421 (2012), both of which are incorporated by reference.

Somatic stem cells can be isolated from their native environment and placed in culture in vitro. One method of isolating SSCs from the primary tissue comprises isolating singe cells from the primary tissue and culturing single cells for clonal expansion. The cells that are capable of clonal expansion are thus identified as SSCs. In one specific embodiment, the cells isolated from the primary tissue can be sorted, prior to culturing, based on size, and the cells that are not the smallest cell isolates from the primary tissue can be cultured and assayed for single cell clonal expansion. In one specific embodiment, the cells that are isolated from the testis that are between about 8-12 μm in diameter are first isolated from cells of other sizes and then assayed for single cell colony expansion. In another embodiment, the cells isolated from primary tissue can be assays for cell markers indicative of SSCs. In specific embodiments, the cell markers that can be used to identify SSCs include but are not limited to SSEA4, GPR125, GFr1α and those listed in Phillips, B. et al., Phil. Trans. R. Soc. B, 365:1663-1678 (2010), which is incorporated by reference in its entirety. In another specific embodiment, a portion of the clonally expanded cells are assayed for markers of SSCs, as disclosed herein, to confirm that the clonally expanded cells are SSCs.

Once isolated and identified as SSCs, the clonally expanded SSCs can be placed in the same cell culture environment in which embryonic stem cells (ESCs) are placed in vitro. Once in this environment, the SSCs will de-differentiate to produce a population of “germline pluripotent stem cells.” These gPSCs display markers of the three embryonic germ layers. Examples of markers of cells that can give rise to the three embryonic germ layers include but are not limited to Oct4, Nanog, Sox2, Lin28, CD73. Markers of specific germ layers are well known in the art and include but are not limited to, Otx1 Otx2 Sox1, nestin, nodal, Wnt genes Sonic hedgehog (SSH) Zic1 as markers of ectoderm, Gata6 Gata4 Sox7 alpha fetal protein (AFP) lefty MixL1 Hnf3b as markers of endoderm, and CDHS FoxF1 fibroblast growth factor (FGF) Brachyury Noggin as markers of mesoderm.

Accordingly, as used herein, gPSCs are a type of pluripotent stem cell that are “derived” from SSCs by removing the SSCs from their native environment and, with or without single cell clonal expansion, placing them in de-differentiation conditions to induce the cells to express markers from all three embryonic germ layers. The de-differentiation conditions can be any environment that can induce the isolated SSCs to de-differentiate into more stem cell-like cells. In one embodiment, the de-differentiation conditions comprise the conditions, e.g., cell culture medium, cell culture conditions and cell culture vessels, in which embryonic stem cells, for example human embryonic stem cells, can be typically cultured. In one embodiment, the de-differentiation conditions comprise culturing the SSCs in cell culture medium for human embryonic stem cells (hESC medium).

The SSCs that are isolated from primary tissue can be from any animal, including but not limited to any mammal, such as mouse, rat, canine, feline, bovine, equine, porcine, non-human and human primates. Mammalian cells particularly suitable for culturing in the culture conditions described herein include SSCs of human origin, which may be cells derived from a testis or ovary. The cells used in the present invention may be normal, healthy cells that are not diseased or not genetically altered. SSCs for initial plating and culturing may be obtained commercially, for example from ATCC (Manassas, Va.), or they may be isolated directly from tissue such that the initial SSCs would represent a primary cell culture.

As used herein, primary SSCs are cells that have been taken directly from living tissue, such as a biopsy, and have not been passaged or only passaged one time. Thus, primary cells have been freshly isolated, often through tissue digestion and plated. Provided the cells have been passaged one time or less, primary cells may or may not be frozen and then thawed at a later time. In addition, the tissue from which the primary SSCs are isolated may or may not have been frozen of preserved in some other manner immediately prior to processing.

When isolating primary cells, tissue should ideally be handled using standard sterile techniques and a laminar flow safety cabinet. In one embodiment, a single needle biopsy is sufficient to isolate enough primary cells to begin the cell culture methods of the present invention. In the case of a tissue biopsy, tissue can be cut into small pieces using sterile instruments. The small pieces can then be washed several times with sterile saline solution or other buffer, such as PBS, that may or may not be supplemented with antibiotics or other ingredients. After washing, the pieces are often, but need not be, treated with an enzymatic solution such as, but not limited to collagenase, dispase or trypsin, to promote dissociation of cells from the tissue matrix.

Dispase is often used to dissociate epithelium from the underlying tissue. This intact epithelium may then be treated with trypsin or collagenase. These digestion steps often results in a slurry containing dissociated cells and tissue matrix. The slurry can then be centrifuged with sufficient force to separate the cells from the remainder of the slurry. The cell pellet can then be removed and washed with buffer and/or saline and/or cell culture medium. The centrifuging and washing can be repeated any number of times. After the final washing, the cells can then be washed with any suitable cell culture medium. Of course, the digestion and washing steps need not be performed if the cells are sufficiently separated from the underlying tissue upon isolation, such as the case in a needle biopsy. Cells may or may not be counted using an electronic cell counter, such as a Coulter Counter, or they can be counted manually using a hemocytometer. Of course, the cells need not be counted at all.

For the purposes of the present invention cells are no longer considered to be primary cells after the cells have been passaged more than once. In addition, cells passaged once or more and immediately frozen after passaging are also considered not to be primary cells when thawed. In select embodiments of the present invention, the SSCs that are initially isolated and cultured are primary cells and, through the use of the methods of the present invention, become non-primary cells after passaging.

By “cell culture” or “culture” is meant the maintenance of the cells in an artificial, in vitro environment. The term “cell culture” also encompasses cultivating individual cells and tissues.

The cells being cultured according to the present invention, whether primary or not, can be cultured and plated or suspended according to the experimental conditions as needed by the technician. The examples herein demonstrate at least one functional set of culture conditions that can be used in conjunction with the methods described herein. If not known, plating or suspension and culture conditions for a given animal cell type can be determined by one of ordinary skill in the art using only routine experimentation. Cells may or may not be plated onto the surface of culture vessels, and, if plated, attachment factors can be used to plate the cells onto the surface of culture vessels. If attachment factors are used, the culture vessels can be precoated with a natural, recombinant or synthetic attachment factor or factors or peptide fragments thereof, such as but not limited to collagen, fibronectin and natural or synthetic fragments thereof.

The cell seeding densities for each experimental condition can be manipulated for the specific culture conditions needed. For routine culture in plastic culture vessels, a seeding density of the gPSCs can be from about 1×10⁴ to about 1×10⁷ cells per cm², which is fairly typical, e.g., 1×10⁶ cells are often cultured in a 35 mm²-100 mm² tissue culture petri dish. Using the methods of the present invention, however, even a single gPSC can be plated or suspended initially. Thus, the methods of the present invention can be performed using 1, 2, 3, 4, 5, 6, 7, 8, 9, 10, 20, 30, 40, 50, 60, 70, 80, 90, 100 or more cells for seeding density. Of course, higher cell seeding numbers can be used, such as but not limited to 1×10³, 1×10⁴, 1×10⁵ and so on. Cell density can be altered as needed at any passage.

Once the gPSCs are generated, they are then placed into a cell culture environment comprising a germline expansion medium (GEM) as described below. Once placed in GEM, the gPSCs can be expanded indefinitely, provided the cells remain in GEM. The expanded gPSCs may or may not lose the ability to express markers from one, two or all three germ lines while being cultured in GEM. In one embodiment, the expanded gPSCs do not express markers from the ectoderm germ layer. In another embodiment, the expanded gPSCs do not express markers from the ectoderm germ layer and/or the mesoderm germ layer. In another embodiment, the expanded gPSCs do not express markers from the ectoderm germ layer and/or the mesoderm germ layer and/or the endoderm germ layer. In another embodiment, the expanded gPSCs do not express markers from the mesoderm germ layer and/or the endoderm germ layer. Once removed from GEM and placed back into ESC medium, however, the gPSCs regain the ability to express at least one marker from all three embryonic germ layers and also regain their pluripotency.

Mammalian cells are typically cultivated in a cell incubator at about 37° C. at normal atmospheric pressure. The incubator atmosphere is normally humidified and often contain about from about 3-10% carbon dioxide in air. Temperature, pressure and CO₂ concentration can be altered as necessary, provided the cells are still viable. Culture medium pH can be in the range of about 7.1 to about 7.6, in particular from about 7.1 to about 7.4, and even more particular from about 7.1 to about 7.3.

Cell culture medium is normally replaced every 1-2 days or more or less frequently as required by the specific cell type. As the gPSCs approach confluence in the culture vessel, they would normally be passaged. As used herein a cell passage is a term that is used as it is in the art and means splitting or dividing the cells and transferring a portion of the cells into a new culture vessel or culture environment. Most likely, the gPSCs used in the methods of the present invention will be adherent to the cell culture surface and will need to be detached. Methods of detaching adherent cells from the surface of culture vessels are well-known and commonly employed and can include the use of enzymes such as trypsin.

A single passage refers to when a technician splits or manually divides the cells one time and transfers a smaller number of cells into a new vessel or environment. When passaging, the cells can be split into any ratio that allows the cells to attach and grow. Thus, at a single passage the cells can be split in a 1:2 ratio, 1:3, 1:4, 1:5 etc. Passaging cells, therefore, is not necessarily equivalent to population doubling. As used herein a population doubling is when the cells divide in culture one time such that the number of cells in culture is approximately doubled. Cells need to be counted to determine if a population of cells has doubled, tripled or multiplied by some other factor. In other words, passaging the cells and splitting them in a 1:3 ratio for further culturing in vitro is not to be taken as the equivalent that the cell population has tripled.

In one embodiment of the present invention, the gPSCs are continuously cultured in vitro. As used herein, “continuous culturing” is the notion that the cells continually divide and reach or approach confluence or a certain density in the cell culture vessel such that the cells require passaging and fresh medium to maintain their health. Thus, the concept of “continuously culturing” is similar to the concept that the gPSCs would be “immortalized.” Accordingly, the term “conditionally immortalized” refers to the ability of the cells to divide in the prescribed culture conditions indefinitely, i.e., regardless of the number of passages, such that the gPSCs growing in the prescribed conditions would need to be passaged to maintain their health. In one embodiment, when cultured using the present methods and conditions of the present invention, normal gPSCs can continue to grow and divide for at least 5, 10, 15, 20, 25, 30, 35, 40, 45, 50, 55, 60, 65, 70, 75, 80, 85, 90, 95, 100, 125, 150, 175, 200, 250 or 300 passages or more.

The present invention is also directed towards methods of stimulating growth of gPSCs in vitro with the methods comprising culturing the gPSCs in the presence of a cell culture medium while inhibiting the activity of ROCK in the gPSCs. Culturing the gPSCs in such conditions will stimulate the gPSCs to grow or proliferate, whereas otherwise the gPSCs may not grow. In one specific embodiment, the cells can grow on plates or in suspension in tight clusters, i.e., the cells become tightly adherent. In another embodiment, the cells grow in suspension and may or may not grow in clusters. In one embodiment, the cultured gPSCs form junctions involving e-cadherin, non-muscle myosin, p120 catenin and gap junction protein such as but not limited to connexin 43 or connexin 36. These types of junctions can be assayed according to Li, D. et al., J. Cell Biol., 191(3):631-644 (2010), which is incorporated by reference.

As used herein and throughout the specification, “cell growth” refers to cell division, such that one “mother cell” divides into two “daughter cells.” As used herein, “cell growth” does not refer to an increase in the actual size of the cells. Stimulation of cell growth can be assayed by plotting cell populations over time. A cell population with a steeper growth curve can said to be growing faster than a cell population with a curve not as steep. Growth curves can be compared for various treatments between the same cell types, or growth curves can be compared for different cell types, e.g., expanded stem cells versus primary stem cells, with the same conditions.

The late passage gPSCs, in particular late passage gPSCs, of the present invention may or may not be characterized by their telomere length. As normally happens, the length of the telomeres generally shortens as cells divide. A cell will normally stop dividing when the average length of telomeres is reduced to a critical length, e.g., 4 kb. In the present invention, the average telomere length of late passage cells may be reduced to a length of as little as 2 kb and continue to grow. The average telomere length is readily determined using routine methods and techniques in the art. Thus in one embodiment, the present invention provides gPSCs capable of dividing in the culture conditions of the present invention, wherein the average telomere length of the gPSCs is shorter than the average telomere length of gPSCs that would normally not divide when placed under different or heretofore routine culture conditions. Thus, the methods of the present invention are capable of generating conditionally immortalized gPSCs whereby the cells have an average telomere length that is less than the average telomere length of gPSCs that are normally capable of dividing and whereby the conditionally immortalized gPSCs are still capable of dividing in spite of their reduced telomere length.

Such currently acceptable or optimal conditions for culturing epithelial cells, including stem cells, generally include culturing cells in well-defined, or synthetic, serum-free medium. For example, culturing gPSCs normally involves culturing in embryonic stem cell (ESC) medium, with or without serum. Thus, “currently acceptable” or “currently optimal” culture conditions include culture conditions where the medium includes serum, such as but not limited to human serum at about 10% and/or serum replacement. Thus the methods of the present invention provide the unexpected results of being able to culture and passage gPSCs for extended periods of time, long after one would have been able to do so using currently acceptable or currently optimal conditions.

As used herein, the term “conditionally immortalized” indicates that the gPSCs may or may not have a reduced average telomere length over the average telomere length of normally expanding gPSCs and are still capable of unlimited growth in the prescribed conditions. The term “conditionally immortalized” can also mean that the gPSCs can grow indefinitely and still retain the ability to express cell markers from all three germ layers when the cells are removed from GEM. In one specific embodiment, “conditionally immortalized gPSCs” are cells that can grow indefinitely in GEM and subsequently regain the pluripotency and the ability to express at least one marker from all three germ layers when placed into ESC culture conditions.

If using telomere length as a measure of conditional immortalization, which is not required for certain embodiments of the present invention, it may be necessary to compare the average telomere length of the conditionally immortalized cells with the average telomere length of non-conditionally immortalized gPSCs that expand normally (slowly) in vitro. The phrase “expand normally” is used to mean a population of gPSCs that, but for being cultured in the conditions outlined herein, would a reduced capacity for rapid expansion in vitro. Therefore, the invention provides methods of conditionally immortalizing gPSCs comprising culturing the gPSCs cells in the presence of a cell culture medium while inhibiting the activity of Rho kinase (ROCK) in the gPSCs during culturing.

The gPSCs can grow, become in need of continuous culturing and/or become conditionally immortalized in vitro without apparent change to the karyotype of the cells after any number of passages. Accordingly, the methods of the present invention comprise continuously culturing gPSCs whereby the cells' karyotype at any passage is not altered or is not substantially altered when compared to the karyotype of primary SSCs or early passage gPSCs. An alteration of a cell's karyotype includes but is not limited to duplication or deletion of chromosomes or portions thereof and/or translocation of a portion of one chromosome to another. Identifying a karyotype and alterations thereof are common techniques in the art. Accordingly, one embodiment of the present invention is directed to late passage gPSCs wherein the late passage gPSCs have (a) an unaltered karyotype when compared to the karyotype of primary SSCs or early passage gPSCs or (b) an unaltered karyotype when compared to the karyotype of initially thawed SSCs or early passage gPSCs. As used herein, a late passage gPSC is defined as a gPSC that has gone through at least 5, 10, 15, 20, 25, 30, 35, 40, 45, 50, 55, 60, 65, 70, 75, 80, 85, 90, 95, 100, 125, 150, 175, 200, 250 or 300 passages or more.

The present invention is also directed to conditionally immortalized gPSCs. In select embodiments, the conditionally immortalized gPSCs, while possibly having an altered phenotype in culture, have (a) an unaltered karyotype when compared to the karyotype of primary SSCs or early passage gPSCs or (b) an unaltered karyotype when compared to the karyotype of initially thawed gPSCs or SSCs

In select embodiments, the methods of the present invention do not use feeder cells. The term “feeder cells” is used herein as it is in the art. Namely, feeder cells are cells that are co-cultured with the “target cells” and share the same medium and vessel as the target cells. The term “feeder cells” is well-known in the art.

In another embodiment, the methods also do not use medium conditioned with feeder cells, i.e., the methods do not use “conditioned medium.” The term conditioned medium is well-known in the art.

The present invention also relates to novel compositions. The novel compositions can be useful for culturing gPSCs. In particular, the cell culture medium used to expand the gPSCs and to conditionally immortalize these cells is referred to as germline expansion medium (GEM).

The cell culture media of the present invention can be any aqueous-based medium and can include any “classic” media such as, but not limited to Dulbecco's Modified Eagle Medium (DMEM) and/or F12 medium. Other cell culture media used in the methods of the present invention include but is not limited to Connaught Medical Research Laboratories (CMRL) 1066 medium (500 ml) supplemented with L-glutamine (5 ml) and 1% Penicillin/Streptomycin (5 ml), 10% human serum (50 ml). The culture medium can also be combinations of any of the classical medium, such as but not limited to CMRL 1066 with and without supplements.

Additional ingredients may be added to the culture medium used in the methods of the present invention. Such additional ingredients include but are not limited to, amino acids, vitamins, inorganic salts, adenine, ethanolamine, D-glucose, heparin, N-[2-hydroxyethyl]piperazine-N′-[2-ethanesulfonic acid] (HEPES), hydrocortisone, insulin, lipoic acid, phenol red, phosphoethanolamine, putrescine, sodium pyruvate, triiodothyronine (T3), thymidine, transferrin and Alk5ii inhibitor. Alternatively, insulin and transferrin may be replaced by ferric citrate or ferrous sulfate chelates. Each of these additional ingredients is commercially available.

Amino acid ingredients which may be included in the media of the present invention include but are not limited to, L-alanine, L-arginine, L-asparagine, L-aspartic acid, L-cysteine, L-glutamic acid, L-glutamine, glycine, L-histidine, L-isoleucine, L-leucine, L-lysine, L-methionine, L-phenylalanine, L-proline, L-serine, L-threonine, L-tryptophan, L-tyrosine and L-valine.

Vitamin that may be added include but are not limited to biotin, choline chloride, D-Ca⁺²-pantothenate, folic acid, i-inositol, niacinamide, pyridoxine, riboflavin, thiamine and vitamin B12.

Inorganic salt ingredients which may be added include but are not limited to calcium salt (e.g., CaCl₂), CuSO₄, FeSO₄, KCl, a magnesium salt, e.g., MgCl₂, a manganese salt, e.g., MnCl₂, sodium acetate, NaCl, NaHCO₃, Na₂HPO₄, Na₂SO₄ and ions of the trace elements selenium, silicon, molybdenum, vanadium, nickel, tin and zinc. These trace elements may be provided in a variety of forms, preferably in the form of salts such as Na₂SeO₃, Na₂SiO₃, (NH₄)6Mo₇O₂₄, NH₄VO₃, NiSO₄, SnCl and ZnSO.

Additional ingredients include but are not limited to heparin, epidermal growth factor (EGF), at least one agent increasing intracellular cyclic adenosine monophosphate (cAMP) levels, and at least one fibroblast growth factor (FGF). Heparin, EGF, the cAMP-increasing agent(s) and FGF(s) may be added to the basal medium or they may be admixed in a solution of, for example, Dulbecco's Phosphate Buffered Saline (DPBS) and stored frozen until being added to basal medium to formulate the medium to be used in the methods of the present invention.

Heparin may be obtained commercially. Heparin is added to the present media primarily to stabilize the activity of the growth factor components, for example FGF. If heparin is used, it may be added to the basal medium at a concentration of about 1-500 U.S.P. units/liter. EGF is available commercially. If EGF is used, it may be added to the basal medium at a concentration of about 0.00001-10 mg/L.

A variety of agents that increase intracellular cAMP levels may be used in formulating the media of the present invention. Included are agents which induce a direct increase in intracellular cAMP levels, e.g., dibutyryl cAMP, agents which cause an increase in intracellular cAMP levels by an interaction with a cellular G-protein, e.g., cholera toxin and forskolin, agents which cause an increase in intracellular cAMP levels by acting as agonists of β-adrenergic receptors, e.g., isoproterenol, and agents which cause an increase in intracellular cAMP levels by inhibiting the activities of cAMP phosphodiesterases, e.g., isobutylmethylxanthine (IBMX) and theophylline. These cAMP-increasing agents are available commercially.

The culture medium used in the methods of the present invention comprises serum or a serum replacement. The serum can be in a concentration (v/v) of from about 1% to about 35%. In select embodiments, the serum is at a concentration of from about 1% to about 20%, or from about 1% to about 15%, or from about 1% to about 10%, or from about 1% to about 5%. If a serum substitute or serum replacement is used, these can be added to the medium according to the manufacturer's suggested protocol. Examples of serum substitutes include but are not limited to commercially available substitutes such as Ultroser™ from Pall Corporation, milk or milk fractions such as but not limited to nonfat dry milk filtrate.

In specific embodiments, the serum used in the methods of the present invention is not bovine or calf serum. In more specific embodiments, the serum used in the methods of the present invention is serum from a primate. In even more specific embodiments, the serum used in the methods of the present invention is human serum.

The range of Ca⁺² concentration used in the embodiments of the present invention can vary. In one embodiment, the concentration of Ca⁺² in the medium used in the methods of the present invention is from 0.1 mM to 10.0 mM. In more specific embodiments, the concentration of Ca⁺² in the medium used in the methods of the present invention can be from about 0.2 mM to about 8 mM, from about 0.4 mM to about 7 mM, from about 0.5 mM to about 5 mM, from about 0.8 mM to about 4 mM, from about 1.0 mM to about 3 mM, from about 1.2 mM to about 2.8 mM, from about 1.4 mM to about 2.6 mM and from about 1.5 mM to about 2.5 mM.

The methods of the present invention comprise inhibiting rho associated coiled-coil protein kinase (ROCK) in the culture. Rho kinase belongs to the Rho GTPase family of proteins, which includes the Rho, Rac1 and Cdc42 kinases. One of the best characterized effector molecule of Rho is ROCK, which is a serine/threonine kinase that binds to the GTP-bound form of Rho. The catalytic kinase domain of ROCK, which comprises conserved motifs characteristic of serine/threonine kinases, is found at the N-terminus. ROCK proteins also have a central coiled-coil domain, which includes a Rho-binding domain (RBD). The C-terminus is made up of a pleckstrin-homology (PH) domain with an internal cysteine-rich domain. The coiled-coil domain is thought to interact with other α-helical proteins. The RBD, located within the coiled-coil domain, interacts only with activated Rho GTPases, including RhoA, RhoB, and RhoC. The pH domain is thought to interact with lipid mediators such as arachidonic acid and sphingosylphosphorylcholine, and may play a role in protein localization. Interaction of the pH domain and RBD with the kinase domain results in an auto-inhibitory loop. In addition, the kinase domain is involved in binding to RhoE, which is a negative regulator of ROCK activity.

The ROCK family currently consists of two members, ROCK1 (also known as ROO or p160ROCK) and ROCK2 (also known as ROKα). ROCK1 is about 1354 amino acids in length and ROCK2 is about 1388 amino acids in length. The amino acid sequences of human ROCK1 and human ROCK2 are well known. For example, the amino acid sequence of ROCK 1 and ROCK2 can be found at UniProt Knowledgebase (UniProtKB) Accession Number Q13464 and 075116, respectively. The nucleotide sequences of human ROCK1 and ROCK2 can be found at GenBank Accession Number NM_005406.2 and NM_004850, respectively. The nucleotide and amino acid sequences of ROCK1 and ROCK2 proteins from a variety of animals are also well-known and can be found in both the UniProt and GenBank databases.

Although both ROCK isoforms are ubiquitously expressed in tissues, they exhibit differing intensities in some tissues. For example, ROCK2 is more prevalent in brain and skeletal muscle, while ROCK1 is more abundant in liver, testes and kidney. Both isoforms are expressed in vascular smooth muscle and heart. In the resting state, both ROCK1 and ROCK2 are primarily cytosolic, but are translocated to the membrane upon Rho activation. ROCK activity is regulated by several different mechanisms, thus Rho-dependent ROCK activation is highly cell-type dependent, ranging from changes in contractility, cell permeability, migration and proliferation to apoptosis. At least 20 ROCK substrates have been identified. See Hu and Lee, Expert Opin. Ther. Targets 9:715-736 (2005) and Loirand et al, Cir. Res. 98:322-334 (2006) and Riento and Ridley, Nat. Rev. Mol. Cell Biol. 4:446-456 (2003) all of which are incorporated by reference.

The role of ROCK in regulating apoptotic signaling is highly cell-type dependent and stimulus dependent. On the other hand, ROCK has also been associated with mediating cell-survival signals in vitro and in vivo. A ROCK-mediated pro-survival effect has been reported in epithelial cells, cancer cells and endothelial cells, as well as in other cell types. In airway epithelial cells, inhibition with Y-27632 or HA 1077 (also known as fasudil) induces membrane ruffling, loss of actin stress fibers and apoptosis (Moore et al., Am. J. Respir. Cell Mol. Biol. 30:379-387, 2004).

Rho/ROCK activation may also play a pro-survival role during oxidative stress-induced intestinal epithelial cell injury (Song et al., Am. J. Physiol. Cell Physiol. 290:C1469-1476, 2006). ROCK has also been associated with pro-survival events in thyroid cancer cells (Zhong et al., Endocrinology 144:3852-3859, 2003), glioma cells (Rattan et al, J. Neurosci. Res. 83:243-255, 2006), human umbilical vein endothelial cells (Li et al., J. Biol. Chem. 277:15309-15316, 2002), hepatic stelate cells (Ikeda et al., Am. J. Physiol. Gastrointest. Liver Physiol. 285:G880-886, 2003) and human neuroblastoma cells (De Sarno et al., Brain Res. 1041: 112-115, 2005). Evidence of ROCK playing a pro-survival role has also been reported in vivo, for example in vascular smooth muscle cells (Shibata et al, Circulation 103:284-289, 2001) and spinal motor neurons (Kobayashi et al, J. Neurosci. 24:3480-3488, 2004).

As used herein, inhibiting ROCK can mean to reduce the activity, function or expression of at least one of ROCK1 or ROCK2. The activity, function or expression may be completely suppressed, i.e., no activity, function or expression, or the activity, function or expression may simply be lower in treated versus untreated cells. In general, ROCK phosphorylates LIM kinase and myosin light chain (MLC) phosphatase after being activated through binding of GTP-bound Rho. One embodiment of the present invention thus involves blocking the upstream pathway of ROCK1 and/or ROCK2, for example GTP-bound Rho, such that ROCK1 and/or ROCK2 is not activated or its activity is reduced over untreated cells. Other upstream effectors include but are not limited to, integrins, growth factor receptors, including but not limited to, TGF-beta and EGFR, cadherins, G protein coupled receptors and the like. Another embodiment of the present invention thus involves blocking the activity, function or expression of downstream effector molecules of activated ROCK1 and/or ROCK2 such that ROCK1 and/or ROCK2 cannot propagate any signal or can only propagate a reduced signal over untreated cells. Downstream effectors include but are not limited to, Myosin phosphatase-targeting protein (MYPT), vimentin, LIMK, Myosin light chain kinase, NHE1, cofilin, Myosin II and the like. For example, both C3 transferase, a ROCK upstream inhibitor that inhibits the activity of Rho, and blebbistatin, a ROCK downstream inhibitor that inhibits the activity of myosin II, when used in the culture conditions described herein in place of a ROCK inhibitor, affected the cells in such a manner as to allow the cells to bypass differentiation and allow proliferation in vitro. Upstream or downstream inhibition of ROCK, in place of direct ROCK inhibition and in conjunction with the other culture conditions described and required herein, may or may not generate conditionally immortalized gPSCs.

The methods of the present invention comprise inhibiting ROCK while culturing the gPSCs. In one embodiment, inhibiting ROCK is accomplished by addition of a ROCK inhibitor to the culture medium. In this embodiment where a ROCK inhibitor is added to culture medium.

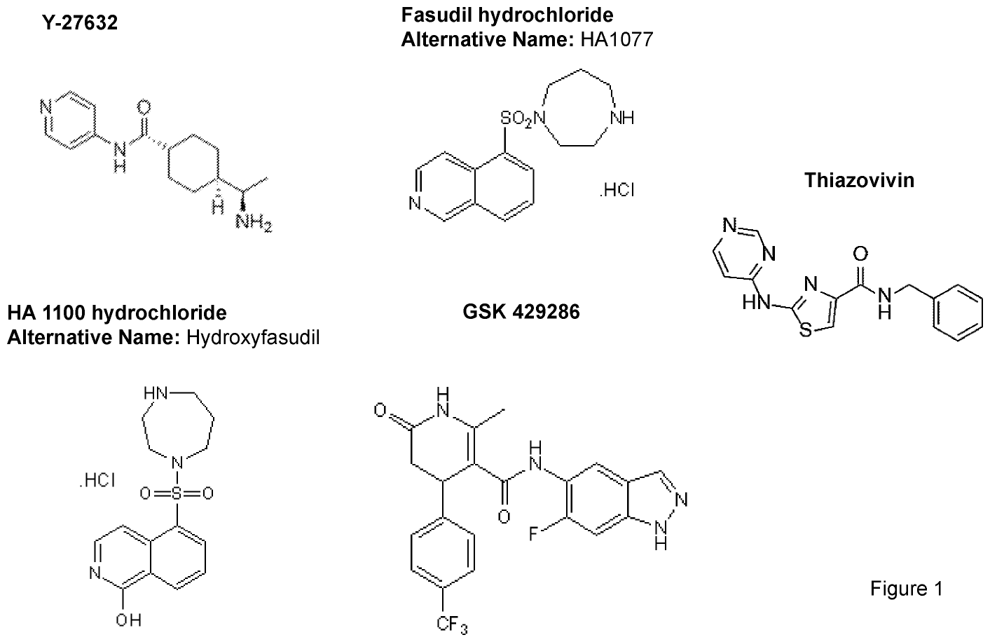

Examples of ROCK inhibitors include but are not limited to Y-27632, HA1100, HA1077, Thiazovivin and GSK429286, the structures of which are depicted in FIG. 1. These compounds are well known and commercially available. Additional small molecule Rho kinase inhibitors include but are not limited to those described in PCT Publication Nos. WO 03/059913, WO 03/064397, WO 05/003101, WO 04/112719, WO 03/062225 and WO 03/062227, and described in U.S. Pat. Nos. 7,217,722 and 7,199,147, and U.S. Patent Application Publication Nos. 2003/0220357, 2006/0241127, 2005/0182040 and 2005/0197328, the contents of all of which are incorporated by reference.

Another way of inhibiting ROCK kinase would be through the use of RNA interference (RNAi). RNAi techniques are well known and rely of double-stranded RNA (dsRNA), where one stand of the dsRNA corresponds to the coding strand of the mRNA that codes for ROCK1, and the other strand is complementary to the first strand. The requirements of optimal RNAi species for a given nucleotide sequence are well-known or can be readily ascertained given the state of the art. For example, it is known that optimal dsRNA is about 20-25nt in length, with a 2 base overhand on the 3′ end of each strand of the dsRNA, often referred to as short interfering RNAs (siRNA). Of course, other well-known configurations such as short hairpin RNA (shRNA) may also work. shRNAs are one continuous RNA strand where a portion is self-complementary such that the molecule is double-stranded in at least one portion. It is believed that the cell processes shRNA into siRNA. The term RNAi molecule, as used herein, is any double stranded double-stranded RNA (dsRNA), where one stand of the dsRNA corresponds to the coding strand of the mRNA that codes for the target gene to be silenced, and the other strand is complementary to the first strand.

Accordingly, one embodiment of the methods of the present invention involves the use of at least one RNAi molecule and/or at least one antisense molecule, to inhibit the activity of ROCK. In one specific embodiment, the RNAi molecule and/or antisense molecule is specific towards ROCK1. In another embodiment, the RNAi molecule or antisense molecule is specific towards ROCK2. In yet another embodiment, the RNAi molecule and/or antisense molecule is specific towards both ROCK1 and ROCK2. In still another embodiment, at least two RNAi molecules and/or antisense molecules are used, where one is specific towards ROCK1 and the other is specific towards ROCK2.

The RNAi molecules and/or antisense molecules may be part of the cell culture by simply soaking the cells with the naked RNAi molecules and/or antisense molecules as has been reported Clemens, J. C., et al., PNAS, 97(12):6499-6503 (2000), which is incorporated by reference. The RNAi molecules and/or antisense molecules may also be part of a complex, such as a liposomal complex that can be used to insert RNAi molecules or antisense/molecules into the cells.

Liposomes fall into two broad classes. Cationic liposomes are positively charged liposomes which interact with the negatively charged dsRNA molecules to form a stable complex. The positively charged dsRNA/liposome complex binds to the negatively charged cell surface and is internalized in an endosome. Due to the acidic pH within the endosome, the liposomes are ruptured, releasing their contents into the cell cytoplasm (Wang et at., Biochem. Biophys. Res. Commun., 1987, 147, 980-985).

Liposomes that are pH-sensitive or negatively-charged entrap dsRNA rather than complex with it. Since both the dsRNA and the lipid are similarly charged, repulsion rather than complex formation occurs. The dsRNA is thus entrapped in the aqueous interior of these liposomes. pH-sensitive liposomes have been used, for example, to deliver dsRNA encoding the thymidine kinase gene to cell monolayers in culture (Zhou et al., Journal of Controlled Release, 1992, 19, 269-274). One major type of liposomal composition includes phospholipids other than naturally-derived phosphatidylcholine. Neutral liposome compositions, for example, can be formed from dimyristoyl phosphatidylcholine (DMPC) or dipalmitoyl phosphatidylcholine (DPPC). Anionic liposome compositions generally are formed from dimyristoyl phosphatidylglycerol, while anionic fusogenic liposomes are formed primarily from dioleoyl phosphatidylethanolamine (DOPE). Another type of liposomal composition is formed from phosphatidylcholine (PC) such as, for example, soybean PC, and egg PC. Another type is formed from mixtures of phospholipid and/or phosphatidylcholine and/or cholesterol. Liposomes that include nucleic acids have been described, for example, in WO 96/40062, U.S. Pat. Nos. 5,264,221, 5,665,710 and Love et al., WO 97/04787 all of which are incorporated by reference.

Another type of liposome, a transfersome, is a highly deformable lipid aggregate which is attractive for drug delivery vehicles. (Cevc et al., 1998, Biochim Biophys Acta. 1368(2): 201-15.) Transfersomes may be described as lipid droplets which are so highly deformable that they can penetrate through pores which are smaller than the droplet. Transfersomes are adaptable to the environment in which they are used, for example, they are shape adaptive, self-repairing, frequently reach their targets without fragmenting, and often self-loading. Transfersomes can be made, for example, by adding surface edge-activators, usually surfactants, to a standard liposomal composition.

Another way ROCK1 and/or ROCK2 RNAi can gain access to the cells in the methods of the present invention is through the use of DNA expression vectors that encode the RNAi molecules and/or antisense molecules. Certain embodiments can utilize only one vector, for example when the RNAi molecule is a shRNA, or when opposing promoters are placed on either side there of the coding sequence for the RNAi molecule. Thus “inhibiting the activity of ROCK” includes the use of DNA that, when transcribed, can block the activity, function or production of ROCK. The liposomal delivery systems described above are one way in which the DNA encoding an RNAi and/or antisense can enter the cell.

Alternatively, the DNA encoding an RNAi and/or antisense can be prepared in a viral vector system that has the capability of entering into cells. These are well-known in the art and include Madzak et al., J. Gen. Virol., 73: 1533-36 (1992) (papovavirus SV40); Berkner et al., Curr. Top. Microbiol. Immunol., 158: 39-61 (1992) (adenovirus); Moss et al., Curr. Top. Microbiol. Immunol., 158: 25-38 (1992) (vaccinia virus); Muzyczka, Curr. Top. Microbiol. Immunol., 158: 97-123 (1992) (adeno-associated virus); Margulskee, Curr. Top. Microbiol. Immunol., 158: 67-93 (1992) (herpes simplex virus (ISV) and Epstein-Barr virus (HBV)); Miller, Curr. Top. Microbiol. Immunol., 158: 1-24 (1992) (retrovirus); Brandyopadhyay et al., Mol. Cell. Biol., 4: 749-754 (1984) (retrovirus); Miller et al., Nature, 357: 455-450 (1992) (retrovirus); Anderson, Science, 256: 808-813 (1992) (retrovirus); C. Hofmann et al., Proc. Natl. Acad. Sci. USA, 1995; 92, pp. 10099-10103 (baculovirus).

In another embodiment, ROCK 1 and/or 2 are inhibited using genetic manipulation techniques, such as, but not limited to, transgenic techniques involving either knockout or dominant negative constructs. Such constructs are disclosed in Khyrul, W., et al., J. Biol. Chem., 279(52):54131-54139 (2004), which is incorporated by reference herein. Other methods of inhibiting ROCK1 and/or 2 using genetic manipulations techniques include RNAi techniques and CRISPR techniques. These techniques and methodologies ware well known in the art.

As mentioned above, one embodiment of blocking ROCK would be to individually or collectively block or inhibit the upstream or downstream effectors molecules of ROCK using any of the methods described herein, such as but not limited to small molecule inhibitors, RNAi techniques, antisense techniques and/or genetic manipulation. Accordingly, any upstream effectors that could be inhibited include but are not limited to, integrins, growth factor receptors, including but not limited to, TGF-beta and EGFR, cadherins, G protein coupled receptors and the like. In addition, any downstream effectors that could be inhibited include but are not limited to, vimentin, LIMK, Myosin light chain kinase, NHE1, cofilin and the like.

In specific embodiments, the novel compositions of the present invention comprise human serum and at least one ROCK inhibitor in a “base” culture medium such as, but not limited to one or more of Minimal Essential Medium (MEM), DMEM, F12, DMEM-F12, RPMI, Leibovitz's L-15, Glasgow Modified Minimal Essential Medium (GMEM), Iscove's Modified Dulbecco's Medium (IMDM) and Eagle's Minimal Essential Medium (EMEM). In additional specific embodiments, the novel compositions of the present invention comprise insulin, human serum and at least one ROCK inhibitor in a “base” culture medium such as, but not limited to one or more of Minimal Essential Medium (MEM), DMEM, F12, DMEM-F12, RPMI, Leibovitz's L-15, Glasgow Modified Minimal Essential Medium (GMEM), Iscove's Modified Dulbecco's Medium (IMDM) and Eagle's Minimal Essential Medium (EMEM). In additional specific embodiments, the novel compositions of the present invention comprise insulin, hydrocortisone, human serum and at least one ROCK inhibitor in a “base” culture medium such as, but not limited to one or more of Minimal Essential Medium (MEM), DMEM, F12, DMEM-F12, RPMI, Leibovitz's L-15, Glasgow Modified Minimal Essential Medium (GMEM), Iscove's Modified Dulbecco's Medium (IMDM) and Eagle's Minimal Essential Medium (EMEM). In additional specific embodiments, the novel compositions of the present invention comprise insulin, hydrocortisone, cholera toxin, human serum and at least one ROCK inhibitor in a “base” culture medium such as, but not limited to one or more of Minimal Essential Medium (MEM), DMEM, F12, DMEM-F12, RPMI, Leibovitz's L-15, Glasgow Modified Minimal Essential Medium (GMEM), Iscove's Modified Dulbecco's Medium (IMDM) and Eagle's Minimal Essential Medium (EMEM). In additional specific embodiments, the novel compositions of the present invention comprise insulin, hydrocortisone, cholera toxin, epithelial growth factor (EGF), human serum and at least one ROCK inhibitor in a “base” culture medium such as, but not limited to one or more of Minimal Essential Medium (MEM), DMEM, F12, DMEM-F12, RPMI, Leibovitz's L-15, Glasgow Modified Minimal Essential Medium (GMEM), Iscove's Modified Dulbecco's Medium (IMDM) and Eagle's Minimal Essential Medium (EMEM).

In additional embodiments, the novel compositions of the present invention comprise CMRL medium supplemented with L-glutamine, 1% Penicillin/Streptomycin, 10% human serum, Alk5ii inhibitor, T3 and B27, which is a commercially available cell culture supplement. CMRL is a commercially available medium that comprises CaCl₂) (anhydrous), KCl, MgSO4 (anhydrous), NaCl, NaH2PO4•H2O, NaHCO3, L-Alanine, L-Arginine•HCL, L-Aspartic Acid, L-Cysteine•HCl•H2O, L-Cystine•2HCl, L-Glutamic Acid, Glycine, L-Histidine•HCl•H2O, Hydroxy-L-Proline, L-Isoleucine, L-Leucine, L-Lysine•HCl, L-Methionine, L-Phenylalanine, L-Proline, L-Serine, L-Threonine, L-Tryptophan, L-Tyrosine•2Na•2H2O, Biotin, Folic Acid, Riboflavin, Ascorbic Acid, D-Ca-Pantothenate, Choline Chloride, knositol, Nicotinic Acid, Nicotinamide, PABA, Pyridoxine•HCl, Thiamine•HCl, Thiamine pyrophosphate:Na, Thymidine, 2′-Deoxyadenosine•H2O, 2′-Deoxycytidine•HCl, 2′-Deoxyguanosine•H2O, 5-Methyl-2′-Deoxycytidine, Uridine-5′-triphosphate•3Na•hydrate, Cholesterol, Polysorbate 80, Coenzyme A Li3 Salt•2H2O, b-NAD•hydrate, b-NADP•Na•4H2O, FAD Disodium Salt, Dextrose, Glutathione (reduced), Sodium acetate, Sodium glucuronate•H2O and L-Glutamine.

The range of concentrations of the supplements can vary. For example the range of L-glutamine between about 0.1% to about 20% (vol glutamine/vol CMRL base), 0.5% to about 15%, 1% to about 10% and about 5% to about 10%. The range of serum can vary from between about 0.1% to about 20% (total vol), 0.5% to about 15%, 1% to about 10% and about 5% to about 10%. The range of Alk5i inhibitor can vary from between about 0.01 mM to about 50 mM, from about 0.1 mM to about 40 mM, from about 1 mM to about 30 mm, from about 5 mM to about 25 mM and from about 10 mM to about 20 mM. The range of T3 can vary from between about 0.001 mM to about 50 mM, from about 0.01 mM to about 40 mM, from about 0.1 mM to about 30 mm, from about 0.5 mM to about 25 mM, from about 1 mM to about 20 mM and from about 5 mM to about 10. The range of B27 can vary from between about 0.01% to about 20% (total vol), from 0.1% to about 15%, from 0.5% to about 10% and from about 1% to about 5%. In one specific embodiment, the novel compositions comprise CMRL medium (500 ml) supplemented with L-glutamine (5 ml), 1% Penicillin/Streptomycin (5 ml), 10% human serum (50 ml), Alk5i inhibitor (10 mM at 1000×) and T3 (1 mM at 1000×).

After culturing in the conditions of the present invention, the cells may be removed from these conditions and placed in a cell culture environment where the environment is absent serum and/or absent another component of GEM, such as but not limited to a ROCK inhibitor. Any combination of one or two of the components of GEM and the ROCK inhibitor may be absent in the subsequent environment. As used herein, a “subsequent environment” when used in connection with a cell culture environment is a cell culture environment in which at least one of the components of GEM is absent. In one embodiment, the ROCK inhibitor is absent in the subsequent environment. In another embodiment, the ROCK inhibitor and serum are absent from the subsequent environment.

In one embodiment, the subsequent environment to the gPSCs, the late passage gPSCs and/or the conditionally immortalized gPSCs is an environment that can promote re-establishment of typical gPSCs and/or does not allow for indefinite proliferation of the gPSCs, the late passage gPSCs and/or the conditionally immortalized gPSCs.

The subsequent environment may also be a “synthetic environment” such that factors known to promote re-establishment in vitro are added to the cell culture. For example, late passage gPSCs, once placed in a subsequent environment that is designed to promote re-establishment of the cells, may begin to form grow in a manner and/or express proteins that resemble mature gPSCs.

In one embodiment, the gPSCs, the late passage gPSCs and or the conditionally immortalized gPSCs are placed into a subsequent environment that is specific to stimulate re-establishment of cells into the gPSCs that grow like and resemble normal gPSCs. Such methods of placing the late passage gPSCs or conditionally immortalized gPSCs in a subsequent environment and promoting or allowing re-establishment of the cells may be referred to herein as “expanding” gPSCs. Accordingly, the population of cells that results from the methods of the present invention are termed herein as “expanded gPSCs.” Various environments for culturing epithelial cells are detailed in Culture of Epithelial Cells (Ian Freshney and Mary G. Freshney, Eds. Wiley-Liss, Inc.) (2^{nd} Ed. 2002), which is incorporated by reference.

In select embodiments, the expanded gPSCs are placed into a subsequent environment that stimulates the cells to differentiate into virtually any cell type present in the animal from which the gPSCs were originally harvested. Examples of cells into which the expanded gPSCs can differentiate include but are not limited to cardiac cells (ventricular, atrial, pacemaker), neural (dopaminergic), pancreatic (alpha, beta, gamma, delta, pp cells), motor neurons, neural crest cells, lung/tracheal cells, epidermal cells, dermal cells, endothelial cells, skeletal muscle cells, bone cells (osteocytes, osteoclasts), retinal cells of the eye, blood cells, liver cells, renal cells, among others.

Alternatively, the cells can be seeded in a subsequent environment into or onto a natural or synthetic three-dimensional cell culture surfaces. One non-limiting example of a three-dimensional surface is a Matrigel®-coated culture surface. Other three dimensional culture environments include surfaces comprising collagen gel and/or a synthetic biopolymeric material in any configuration, such as but not limited to a hydrogel. Of course, a variety of three dimensional culture surfaces may be used simultaneously with the methods the present invention. These three-dimensional cell culture surface environments may or may not promote re-establishment.

In one embodiment, the late passage gPSCs and/or the conditionally immortalized gPSCs can be genetically modified to express a protein of interest. The genetic modification of the cells would not be a modification designed to immortalize the cells, such as the insertion of a viral protein. Rather, the genetic modification of the cells would be designed to, for example, insert a transgene that codes for a protein. For example, once gPSCs are isolated and expanded using the cell culture methods of the present invention, the cells can subsequently be manipulated and a transgene coding for a protein, including but not limited to, a marker protein, can be inserted in the genome of the cells. These cells can then be placed in a subsequent environment, such as an autologous implant into a subject, such that the cells will produce the protein encoded by the transgene.

The methods by which the transgenes are introduced into the cells are standard methods known from the literature for in vitro transfer of DNA into mammalian cells, such as electroporation; calcium phosphate precipitation or methods based on receptor-mediated endocytosis, disclosed in WO 93/07283, which is incorporated by reference. Other methods and materials for inserting a gene of interest into cells are disclosed in Sambrook et al., Molecular Cloning: A Laboratory Manual, Cold Springs Harbor Laboratory Press, Third Edition (2001), which is incorporated by reference.

A wide variety of genes of interest can be expressed in the gPSCs, the late passage gPSCs and/or the conditionally immortalized gPSCs. These genes of interest include, but are not limited to, sequences encoding toxins, enzymes, prodrug converting enzymes, antigens which stimulate or inhibit immune responses, tumor necrosis factors, cytokines, and various proteins with therapeutic applications, e.g., growth hormones and regulatory factors and markers, such as green fluorescent protein and the like.

After transfecting the gPSCs, the late passage gPSCs and/or the conditionally immortalized gPSCs of the present invention, these cells that were successfully transfected can be selected for using markers that are well known in the art. After selection of the successfully transfected cells, the genetically modified gPSCs, the late passage gPSCs and/or the conditionally immortalized gPSCs of the present invention can be cultured using the cell culture techniques of the present invention to produce a population of genetically modified gPSCs, late passage gPSCs and/or conditionally immortalized gPSCs. These cells can subsequently be collected and placed into a subsequent environment as described above, including but not limited to being placed back into the subject, i.e., an autologous implant.

The present invention also provides kits for culturing gPSCs and/or generating conditionally immortalized gPSCs. The kits can include culture vessels, culture media in wet or dry form and/or individual media components such as serum. The kit may or may not include chemicals, such as trypsin, for passaging cells, etc.

### EXAMPLES

### Example 1—Isolation of SSCs from Human Testes

Recent investigations have demonstrated that when isolated and cultured in the proper medium, germline stem cells can be induced to form cell/tissue from all three germ layers, i.e., ectoderm, mesoderm, and endoderm. These cells have been named using different acronyms including hgPSCs and hESLCs.

The tunica albica was removed and the seminiferous tubules were cut from testes into 1 g tissue samples and either stored in liquid nitrogen or used fresh.

A 10 ml enzyme solution of 1× Hank's Balanced Salt Solution (HBSS) was prepared with 2.5 mg/ml collagenase, 1.25 mg/ml dispase. The solution can also be used on frozen testis tissue samples during the isolation process. Frozen tissue samples were transferred to a 120 ml container with 40 ml ice-cold DMEM/F12+Antibiotic-Antimycotic, and washed twice. After washing in the medium, 2-3 ml of the medium was left in the 120 ml container (on ice) where the sample tissue was sliced with sterile scissors. The tissue was transferred into a 50 ml tube with an additional 40 ml ice-cold DMEM/F12+Antibiotic-Antimycotic. The tissue was allowed to sediment for 2-5 minutes and supernatant was removed and washed with the enzyme solution. The enzyme solution then settled and incubated 30 min in a 37° C. water bath with 100 rpm shaking. Afterwards, the enzyme was removed and re-suspended in 10 ml hESC medium (DMEM/F12 500 ml, knockout serum replacement 100 ml, non-essential amino acids 5 ml, L-glutamine 5 ml, and Antibiotic-Antimycotic 5 ml). A 40 μm mesh filter was placed atop of a 50 ml tube and the supernatant and sample were slowly filtered through the 40 μm cell strainer mesh to extract spermatogonial cells. The filtered tissue sampled was then centrifuged (1000 rpm/5 min). The supernatant was removed, and re-suspended in fresh 6 ml hESC medium. The medium and sample were then seeded into a 6-well uncoated tissue culture plate. Lastly, 3.5 μl of 10 ng/ml GDNF was placed in the sample wells and the plate was placed into a 34° C. and 5% CO₂ incubator to start the cell culturing process.

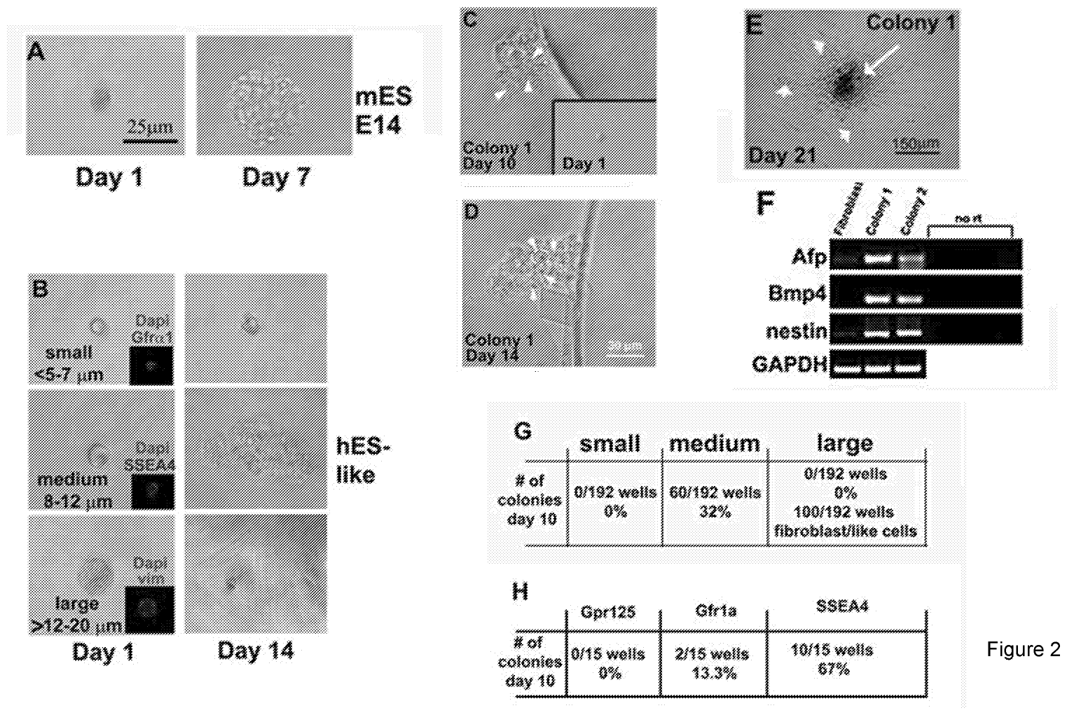

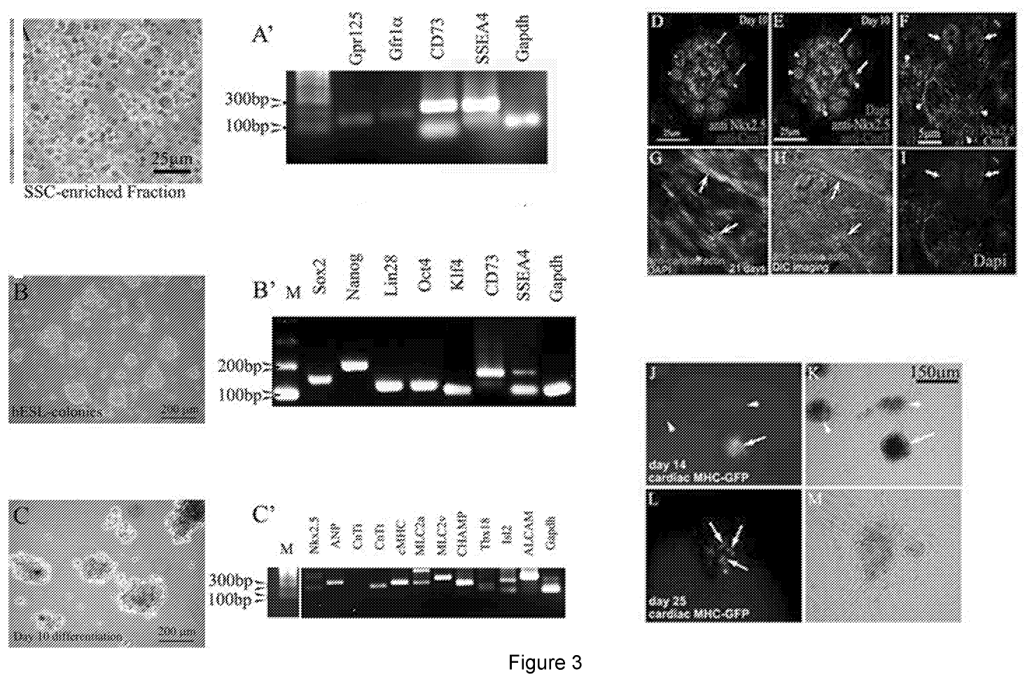

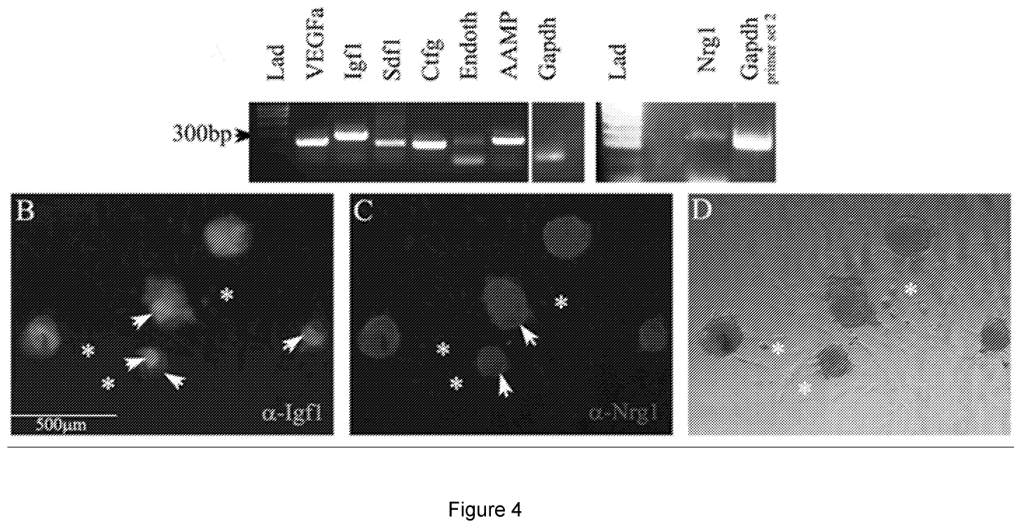

To begin identifying this stem cell population, single cell suspensions were formed and assayed for their ability to undergo clonal expansion, which is accepted as a primary characteristic of stem cells. Cell size was used as a marker for isolating and identifying the population of SSCs. FIG. 2 shows typical examples of isolated single cells, while FIG. 2A shows the milieu of different cell sizes immediately after enzymatic isolation from a testis. Using a hand-drawn glass pipette connected to a plastic filtered mouth suctioning tip, small (<5-7 μm), medium (˜8-12 μm), and large (>12-20 μm) cells were placed one by one into wells of a 96 well plate. A typical, single mouse ESC cell expands into a colony very quickly after plating. Not all cells isolated from the human testis expanded in vitro. After 10-14 days of culture, only medium-sized cells grew into colonies that were capable of differentiating and expressing markers from the three embryonic germ layers (FIG. 2C-F).

To apply a more rigorous approach for identifying which cell type could grow clonally into colonies of gPSCs, the cells were assayed for SSEA4 expression as a candidate marker for the SSCs that might give rise to gPSCs. Cells were labeled with primary antibodies directed against SSEA4, Gfra1, Gpr125, or vimentin, followed by fluorescently labeled secondary antibodies. The insets in FIG. 1B show examples of fluorescent single cells within each well. Interestingly, SSEA4+ cells not only formed colonies, they were the same size as the medium cells previously seen to form colonies. Conversely, Gfrα1+ cells were much smaller and did not form colonies while the larger cells were only able to divide a few times in culture and resembled fibroblasts. Most of the larger cells were vimentin positive, suggesting they were Sertoli cells or cells from the lamina propria.

### Example 2—Production of gPSCs from SSCs

After isolation, SSCs were cultured in DMEM 20% serum replacement medium (hESC medium) along with 3.5 μl of GDNF for 4 days to stimulate growth and colony formation. The cells were incubated at 37° C. and 5% CO₂. Media was change every other day. After the 4th day of incubation, the hESC medium plus GDNF was replaced with hESC medium supplemented with 4 ng/ml basic fibroblast growth factor (bFGF). Colonies were cultured for at least 10 days to form the initial populations of gPSCs.

### Example 3—Continuous Culturing of gPSCs

The initial population of established gPSCs were expanded in germline expansion medium (GEM: complete DMEM high glucose, Ham's F12 nutrient mixture, 0.13 μg/ml hydrocortisone, 5 mg/ml insulin, 11.7 μM choleratoxin, 10 mg/ml gentamycin 10 mg/ml) containing 5 mM ROCK inhibitor (Y-27632) for 7-10 days. J2 cells were not used. Fetal bovine serum was replaced with human serum to remove all animal products.

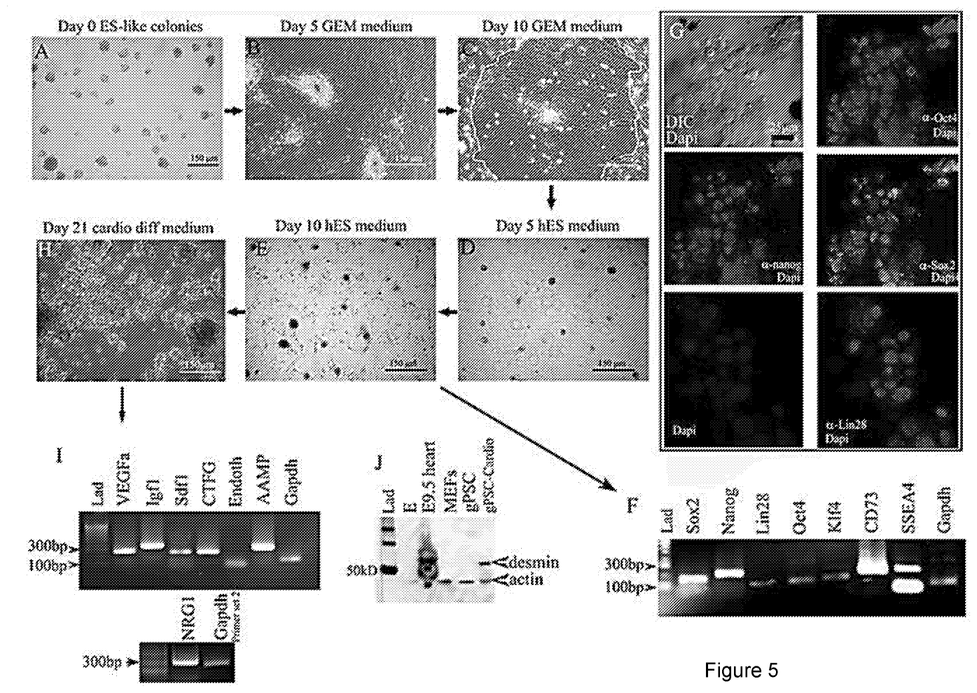

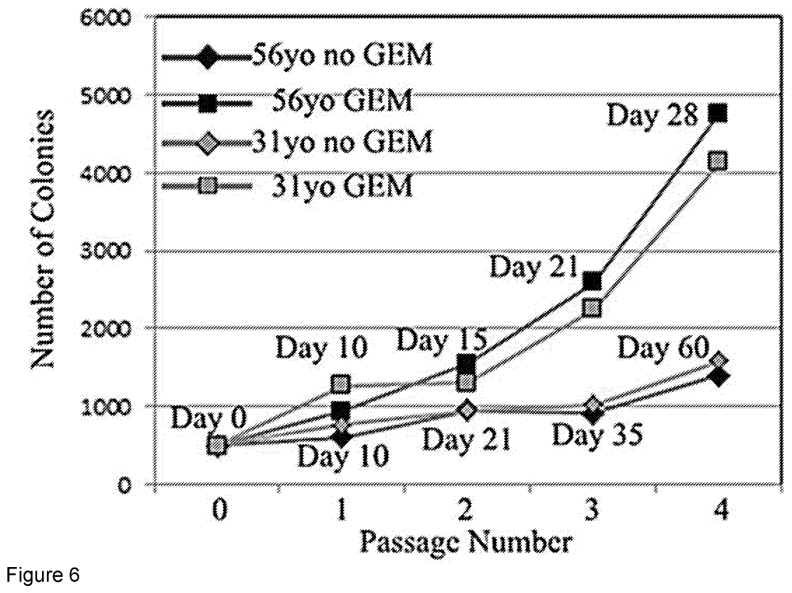

FIG. 5 illustrates the process of germ cell expansion and subsequent re-establishment of hgPSCs, followed by their differentiation into cardiac lineages. By day 10 in GEM, the cells took on a cobble-stone appearance. While in GEM, these cells could be expanded indefinitely and quickly, e.g., one colony placed in a 96 well plate can typically be split into two wells of a 96 well plate within 7 days of plating. Expansion rates of the continuously cultured gPSCs grown in GEM were compared to expansion rates using convention culture method systems. After 4 passages, the continuously cultured gPSCs were re-established in hESC medium, and this resulted in ˜4× more colonies compared to gPSCs expanded in conventional culture conditions. More importantly, it took 30 days less time to expand gPSCs in GEM compared to gPSCs grown in conventional conditions.

### Example 4—Re-Establishing gPSCs from Expansion Cultures

After expansion in GEM, the medium was replaced with hESC medium containing bFGF. This medium was replaced every other day. Colonies resembling hESC colonies spontaneously and consistently formed within 5-10 days.

gPSCs have been expanded in GEM to amounts of at least 20×. Re-establishing gPSC colonies can be accomplished by replacing GEM with hESC medium. Usually within ten days of culturing in GEM, gene expression patterns match primary gPSCs including the expression of all Yamanaka factors, nanog, and CD73.

### Example 5—Differentiation of gPSCs into Cardiac Lineages

Unexpanded or previously expanded gPSCs were cultured in hESC medium containing 20% serum replacement and 0.25 μM Cardiogenol-C for 10 days. After differentiation, the media was replaced with DMEM medium supplemented with 20% human serum. This medium was considered post-differentiation medium where the cardiac clusters can be cultured for up to 30 days.

The reestablished gPSC colonies were differentiated down the cardiac pathway resulting in an expression pattern of paracrine factors similar to that observed in cardiac cells differentiated from primary hgPSCs (FIG. 5H, 5I).

While it was important to show that cardiomyocytes derived from hgPSCs expressed cardiac and paracrine factor genes it was further necessary to identify their physiological ability to secrete those paracrine factors. A consensus of at least six paracrine effects categories is well-established (see Table 1). These categories include (Survival, proliferation, immune cells, remodeling, vascularization, and CPC activation).

TABLE 1

| | Abbreviation | BP Size | | Published function |
|---|---|---|---|---|
| SSC GENE | | | | |
| CANDIDATES | | | | |
| G protein coupled | GPR125 | 136 | BPS | Canidate marker for human and mouse |
| receptor 125 | | | | SSCs. Multiple functions in different tissues. |
| GDNF family | GFR1A | 163 | BPS | Canidate marker for human and mouse |
| receptor 1 alpha | | | | SSCs. |
| Stage-specific | SSEA-4 | 359 | nps | Expressed in specific cells of the |
| antibody-4 | | | | seminiferous basal membrane. Canidate |
| | | | | marker for human and mouse SSCs. |
| hESC GENES | | | | |
| SRY (Sex | SOX2 | 153 | bps | Transcriptional regulator of somatic cell |
| Determining | | | | reprogramming; helps maintain |
| Region Y)- Box 2 | | | | pluripotency. |
| Octamer-Binding | OCT3/4 | 117 | bps | Plays the central role in pluripotency. |
| Transcription | | | | |
| Factor 3/4 | | | | |
| LIN28 Homolog A | LIN 28A | 128 | bps | Regulates stemness and self renewal |
| | | | | capacity in human and mouse pluripotent |
| | | | | stem cells. |
| Homeobox | NANOG | 200 | bps | Required for final states of pluripotency. |
| Transcription | | | | |
| Factor Nanog | | | | |
| Kruppel-like factor | KLF-4 | 143 | bps | Essential for ESC maintenance and self- |
| 4 | | | | renewal capacity. |
| Cluster of | CD73 | 219 | bps | Essential stemness marker for human |
| differentiation 73 | | | | somatic cells. |
| EARLY CARDIAC | | | | |
| GENES | | | | |
| Activated | ALCAM | 388 | bps | Surface marker for early cardiomyocytes |
| Leukocyte Cell | | | | |
| Adhesion | | | | |
| Molecule | | | | |
| Cardiac Helicase | CHAMP | 200 | bps | Cardiac transcription factor expressed |
| Activated MEF2C | | | | specifically in postnatal and embryonic |
| protein | | | | cardiomyocytes. |
| T-Box | TBX18 | 112 | bps | It is critical for early sino atrial node (SAN) |
| Transcription | | | | specification (pacemaker cells). |
| Factor 18 | | | | |
| DIFFERENTIATED | | | | |
| CARDIAC GENES | | | | |
| Atrial Natriuretic | ANP | 204 | bps | Cardiac hormone that regulates blood |
| Peptide | | | | pressure, vasodilation, natriuresis, and |
| | | | | diuresis. |
| NK2 homeobox 5 | NKX2.5 | 215 | bps | Cardiac transcription factor responsible for |
| | | | | heart formation and development. |
| Cardiac Troponin-I | CTNI | 335 | bps | Key regulatory protein associated with the |
| | | | | thin filament, inhibits actomyosin |
| | | | | interactions at diastolic levels of |
| | | | | intracellular Ca2+. |
| Cardiac Troponin- | CTNT | 217 | bps | Fixation of troponin complex on the actin |
| T | | | | filament and also participitates in muscle |
| | | | | contraction |
| Myosin heavy | MHC | 542 | bps | Molecular motor of the heart that |
| chain | | | | generates motion by coupling its ATPase |
| | | | | activity to its cyclic interaction with actin. |
| Myosin light chain | MLC2A | 270 | bps | Atrial marker expressed during devlopment |
| 2A | | | | and adulthood. Also regulates heart |
| | | | | contraction along with MLC 2V. |
| Myosin light chain | MLC2V | 380 | bps | Ventricular marker during human heart |
| 2V | | | | development and in adulthood |
| Myocyte Enhancer | ISL-1 | 127 | bps | a LIM homeodomain transcription factor |
| Factor 2C | | | | expressed in majority of cells in both right |
| | | | | ventricle and atria of the heart. ISL1 is also |
| | | | | responsible for survival, proliferation, and |
| | | | | migration of cardiac progenitor cells. |
| PARACRINE | | | | |
| FACTORS | | | | |
| Vascular | VEGFA | 280 | bps | Promotes vasculo-and angiogenesis in |
| Endothelial | | | | myocardium and cardiomyocyte |
| growth factor-A | | | | proliferation. Also helps regenerates |
| | | | | mycoardium. |
| Insulin-like growth | IGF-1 | 372 | bps | Switch macrophages from pro-inflammatory |
| factor | | | | to anti-inflammatory phenotype both in |
| | | | | vitro and vivo. Also promotes resident stem |
| | | | | mobilization and cardiac lineage |
| | | | | commitment. |
| Stromal derived | SDF-1 | 250 | bps | Promotes repair and regeneration by |
| factor-1 | | | | recruiting circulating progenitor cells to the |
| | | | | injured site. Also secreted by cardiac stem |
| | | | | cells. |
| Connective tiddue | CTGF | 237 | bps | Acts as a cofactor for other grotwth factor |
| growth factor | | | | that promotes fibrosis and wound healing |
| | | | | by enhancing ECM protein synthesis. |
| Endothelin-1 | END-1 | 270 | bps | Vasoactive peptide secreted by the |
| | | | | endothelium required for cardiomyocyte |
| | | | | survival. It decreases susceptibility to TNF- |
| | | | | mediated apoptosis; secreted from |
| | | | | cardiomyocytes under mechanical stress. |
| Angio-associated | AAMP | 283 | bps | Associated with angiogenesis, endothelial |
| migratory protein | | | | tube formation, and migration of |
| | | | | endothelial cells. It may also regulate |
| | | | | smooth muscle cell migration via the RhoA |
| | | | | pathway. |
| Neuregulin-1 | NRG-1 | 203 | bps | Activates ErbB2 receptor present on |
| | | | | differentiated cardiomyocytes and |
| | | | | promotes cardiomyocyte proliferation. |
| Indoleamine 2,3- | IDO | 222 | bps | Inhibits T-cell and NK cell proliferation, |
| dioxygenase | | | | cytotoxicity, cytokine production; also |
| | | | | mediates T-cell apoptosis. |

TABLE 2
CARDIOMYOCYTE PRIMER SEQUENCES (5'-3')

| | Abbreviation |
|---|---|
| SSC GENE CANDIDATES | |
| F- AAAGCTTGGCGCAGATGTGA | GPR125 |
| R-TTGCCACGGCATTGGTAAGA | |
| F-TTTACCAACTGCCAGCCAGA | GFR1A |
| R-TGTTGCTGCAGTCACACCAT | |
| F-GAGAAGCTGTTCCAGATAGTGC | SSEA-4 |
| R-CTCAGGGTACATGAAATGGTGG | |
| hESC GENES | |
| F-ATGTACAACATGATGGAGACGG | SOX2 |
| R-CCACACCATGAAGGCATTCA | |
| F-TTTGCCAAGCTCCTGAAGCA | OCT3/4 |
| R-AAAGCGGCAGATGGTCGTTT | |
| F-GAGCATGCAGAAGCGCAGATCAAA | LIN 28A |
| R-TATGGCTGATGCTCTGGCAGAAGT | |
| F-TCAGAGACAGAAATACCTCAGC | NANOG |
| R-AGGAAGAGTAAAGGCTGGGG | |
| F-TTCAACCTGGCGGACATCAA | KLF-4 |
| R-TTCAGCACGAACTTGCCCAT | |
| F-GTATTGCCCTTTGGAGGCAC | CD73 |
| R-AGGGTCATAACTGGGCACTC | |
| EARLY CARDIAC GENES | |
| F-TTCCAGAACACGATGAGGCA | ALCAM |
| R-ACCTGTGACAGCTTGGTAGA | |
| F-AAGGTGTCTAGTAAGACAGCAG | CHAMP |
| R-ATCATTTTGCCTAGCCCACC | |
| F-ATGCATTCTGGCGACCATCA | TBX18 |
| R-ACGCCATTCCCAGTACCTTG | |
| DIFFERENTIATED CARDIAC GENES | |
| F-AGTGGATTGCTCCTTGACGA | ANP |
| R-GGGCACGACCTCATCTTCTA | |
| F-ACCCTAGAGCCGAAAAGAAAG | NKX2.5 |
| R-GCCGCACAGTAATGGTAAGG | |
| F-AAGATCTCCGCCTCGAGAAA | CTNI |
| R-GCAGAGATCCTCACTCTCCG | |
| F-CTTTGATGAGAGACGTCGGG | CTNT |
| R-CTTCCCACTTTTCCGCTCTG | |
| F-GGGGACAGTGGTAAAAGCAA | MHC |
| R-TCCCTGCGTTCCACTATCTT | |
| F-GAGTTCAAAGAAGCCTTCAGC | MLC2A |
| R-ATCCTTGTTCACCACCCCTT | |
| F-GGTGCTGAAGGCTGATTACG | MLC2V |
| R-TTGGAACATGGCCTCTGGAT | |
| F-CTGTGGGCTGTTCACCAACT | ISL-1 |
| R-GCCGCAACCAACACATAGG | |
| PARACRINE FACTORS | |
| F-GGGCAGAATCATCACGAAGT | VEGFA |
| R-TGTTGTGCTGTAGGAAGCTC | |
| F-GAGCCTGCGCAATGGAATAA | IGF-1 |
| R-ATACCCTGTGGGCTTGTTGA | |
| F-TCAACACTCCAAACTGTGCC | SDF-1 |
| R-AGCAAGTGAACTGTGGTCCAT | |
| F-CGACTGGAAGACACGTTTGG | CTGF |
| R-TTTGGGAGTACGGATGCACT | |
| F-CACAACAGAGCCAACAGAGTC | END-1 |
| R-TCCAGGTGGCAGAAGTAGAC | |
| F-AGAGTGAGTCCAACTCGGTG | AAMP |
| R-AGGGCAAAGTCCAGGATCTC | |
| F-GGAGCATATGTGTCTTCAGCTAC | NRG-1 |
| R-AAGCTGGCCATTACGTAGTTTTG | |

TABLE 3

| | 56 yo Patient 1 | | | | | | | | | | | | | | | | |
|---|---|---|---|---|---|---|---|---|---|---|---|---|---|---|---|---|---|
| Passage | Number of gPSC colonies | | | | | | | | | | | | | | | | |
| # | NO Conditional Reprogramming Medium (No GEM) | | | | | | | | | | | | | | | | Time |
| P0 | 500 | | | | | | | | | | | | | | | | Day 0 |
| P1 | 380 | | | | | | | | 320 | | | | | | | | Day 10 |
| P2 | 280 | | | | 220 | | | | 235 | | | | 215 | | | | Day 21 |
| P3 | 113 | | 163 | | 89 | | 108 | | 123 | | 93 | | 105 | | 113 | | Day 35 |
| P4 | 85 | 94 | 101 | 96 | 58 | 73 | 80 | 93 | 103 | 94 | 76 | 89 | 88 | 91 | 87 | 92 | Day 60 |

| Total # | 1,400 | | | | | | | | | | | | | | | | |
|---|---|---|---|---|---|---|---|---|---|---|---|---|---|---|---|---|---|
| colonies | | | | | | | | | | | | | | | | | |
| | 56 yo Patient 1 | | | | | | | | | | | | | | | | |
| Passage | Number of gPSC colonies | | | | | | | | | | | | | | | | |
| # | Expanded in Conditional Reprogramming Medium (+GEM) | | | | | | | | | | | | | | | | Time |
| P0 | 500 | | | | | | | | | | | | | | | | Day 0 |
| P1 | 455 | | | | | | | | 476 | | | | | | | | Day 10 |
| P2 | 387 | | | | 358 | | | | 396 | | | | 403 | | | | Day 15 |
| P3 | 308 | | 310 | | 303 | | 317 | | 302 | | 287 | | 363 | | 397 | | Day 21 |
| P4 | 302 | 275 | 280 | 285 | 295 | 283 | 301 | 275 | 267 | 258 | 275 | 285 | 355 | 310 | 365 | 352 | Day 28 |

| Total # | 4,763 | | | | | | | | | | | | | | | | |
|---|---|---|---|---|---|---|---|---|---|---|---|---|---|---|---|---|---|
| colonies | | | | | | | | | | | | | | | | | |
| | 31 yo Patient 2 | | | | | | | | | | | | | | | | |
| Passage | Number of gPSC colonies | | | | | | | | | | | | | | | | |
| # | NO Conditional Reprogramming Medium (No GEM) | | | | | | | | | | | | | | | | Time |
| P0 | 500 | | | | | | | | | | | | | | | | Day 0 |
| P1 | 385 | | | | | | | | 380 | | | | | | | | Day 10 |
| P2 | 286 | | | | 222 | | | | 205 | | | | 235 | | | | Day 21 |
| P3 | 133 | | 114 | | 113 | | 124 | | 143 | | 127 | | 118 | | 147 | | Day 35 |
| P4 | 101 | 102 | 88 | 98 | 84 | 88 | 104 | 102 | 110 | 113 | 95 | 101 | 95 | 87 | 115 | 103 | Day 60 |

| Total # | 1.587 | | | | | | | | | | | | | | | | |
|---|---|---|---|---|---|---|---|---|---|---|---|---|---|---|---|---|---|
| colonies | | | | | | | | | | | | | | | | | |
| | 31 yo Patient 2 | | | | | | | | | | | | | | | | |
| Passage | Number of gPSC colonies | | | | | | | | | | | | | | | | |
| # | Expanded in Conditional Reprogramming Medium (+GEM) | | | | | | | | | | | | | | | | Time |
| P0 | 500 | | | | | | | | | | | | | | | | Day 0 |
| P1 | 489 | | | | | | | | 478 | | | | | | | | Day 10 |
| P2 | 287 | | | | 318 | | | | 337 | | | | 306 | | | | Day 15 |
| P3 | 221 | | 247 | | 276 | | 285 | | 307 | | 315 | | 287 | | 305 | | Day 21 |
| P4 | 205 | 210 | 222 | 230 | 245 | 237 | 275 | 245 | 301 | 275 | 303 | 289 | 275 | 263 | 300 | 268 | Day 28 |

| | |
|---|---|
| Total # | 4,143 |
| colonies | |

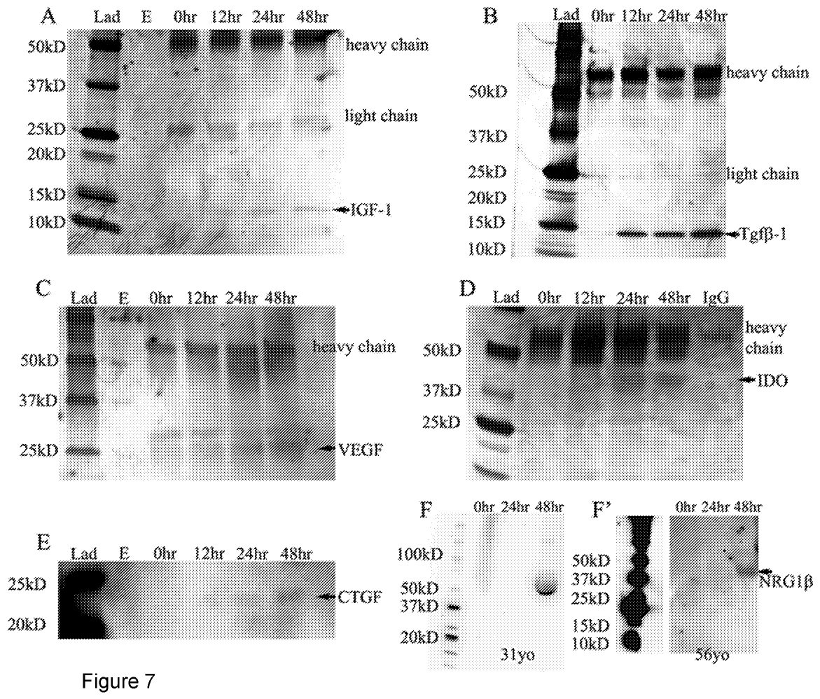

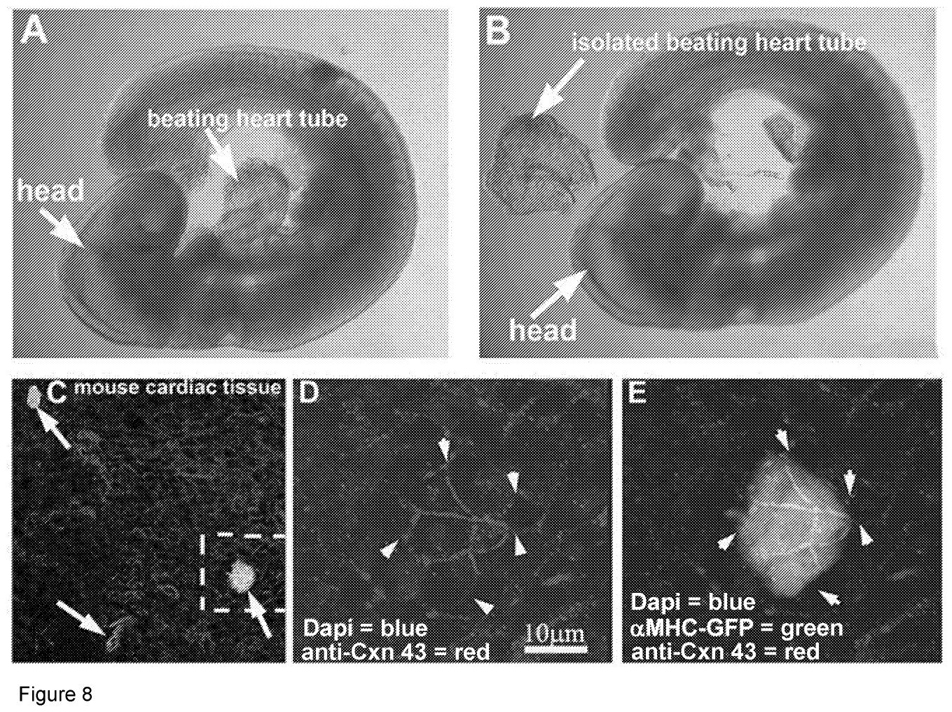

To detect secretion, two different methods were employed. First, medium (1.0 ml) was directly tested by immunoprecipitation (IP) using antibodies to specific paracrine factors, followed by antibody isolation using magnetic protein G-coated beads. The pull-down products were subjected to SDS-PAGE, followed by silver stain of the gels. This highly sensitive approach revealed that VEGFA, CTGF, IGF-1, and TGFβ all increased in expression over a 48 hr period of incubation (FIG. 7).

Second, proteins were precipitated from the medium using 4 volumes of ice cold ethanol and then separated by SDS-PAGE. The isolated proteins were probed via Western blot using antibodies directed against each paracrine factor (FIGS. 7E and 7F). This Western blot approach was employed primarily where the antibodies had not been tested for immunoprecipitation applications. Here, it was found that CTGF and NRG-1 were secreted into the medium by 24-48 hrs. When combined, these data provided good evidence that gPSC-derived cardiomyocytes secrete paracrine factors known to be proangiogenic and procardiogeneic.

Cardiac-specific, GFP-labeled, gPSC-derived cardiomyocytes were transplanted into fetal heart tissue to determine if the gPSC-derived cardiomyocytes could fuse with beating heart tissue. Many different animal models have been utilized to demonstrate infiltration and integration-ability of various types of stem cells within cardiac tissue. Using fetal mouse heart tissue is generally a better model than adult cardiac model systems because the mouse fetal heart beats at ˜60-70 beats/min, which is very similar physiologically to the human heart.

cMHC-GFP-positive gPSC-derived cardiac colonies were physically isolated by mouth pipette using a Leica Fluorescent stereoscope. GFP+ Colonies were then pipetted into tight crevices within the heart tube so they could not fall away from the beating heart tube. Each well of the 96 well plate contained 10-15 fetal hearts, which forced virtually all loose cardiac colonies to remain in close contact with cardiac tissue. After 48 hrs of incubation, fetal hearts were observed live using the same fluorescent stereoscope to identify GFP(+) areas. Upon detection, fetal hearts were fixed in 3.0% formaldehyde for 2 hrs, followed by processing for immunofluorescence using an antibody for the cardiac gap junction protein Connexin 43 (CNX43). Multiple areas of GFP-labeled cells were visibly co-stained with the gap junction protein CNX43 providing evidence that the hgPSC-derived cardiac cells directly fused with the mouse cardiac tissue. Fusion of GFP labeled cells was observed in 8 out of the 10 fetal hearts.

The Examples of Embodiments disclosed herein are meant to be illustrative and are not intended to limit the scope of the present invention in any manner.

What is claimed is:

1. A method of continuously culturing germline pluripotent stem cells (gPSCs), the method comprising
a) culturing the cells in the presence of a cell culture medium, and
b) inhibiting the activity of Rho kinase (ROCK) during culturing.

2. The method of claim 1, wherein the gPSCs are human cells.

3. The method of claim 1, wherein the gPSCs are not primary cells.

4. The method of claim 1, wherein the gPSCs were derived from spermatogonial stem cells or ovarian stem cells.

5. The method of claim 1, wherein the cell culture medium comprises serum or a serum replacement.

6. The method of claim 5, wherein the serum is human serum.

7. The method of claim 1, wherein the ROCK is Rho kinase inhibitor 1 (ROCK 1), Rho kinase inhibitor 2 (ROCK 2) or both.

8. The method of claim 1, wherein inhibiting the activity of ROCK comprises culturing the gPSCs in the presence of a small molecule ROCK inhibitor.

9. The method of claim 8, wherein the small molecule ROCK inhibitor is selected from the group consisting of Y-27632, HA1100 hydrochloride, HA1077 and GSK429286.

10. The method of claim 8, wherein inhibiting the activity of ROCK comprises culturing the gPSCs in the presence of an RNA interference (RNAi) molecule specific for ROCK 1, ROCK 2 or both.

11. The method of claim 1, further comprising
c) passaging the gPSCs after inhibiting ROCK, and
d) placing the passaged cells in cell culture environment in which ROCK is not being inhibited.

12. The method of claim 11, wherein the cell culture environment in which ROCK is not being inhibited is a three-dimensional cell culture environment.

13. The method of claim 11, wherein the cell culture environment in which ROCK is not being inhibited induces the gPSCs to differentiate into at least one differentiated cell type.

14. The method of claim 13, wherein the at least one differentiated cell type is a cardiomyocyte.

15. A method of implanting cardiomyocytes into the heart of a subject in need thereof, comprising the transplanting cardiomyocytes that are produced by the method of claim 14.

16. The method of claim 15, wherein the subject has a heart defect.

17. A population of conditionally immortalized germline pluripotent stem cells (igPSCs).

18. The cell population of claim 17, wherein the conditionally igPSCs are derived from spermatogonial stem cells or ovarian stem cells.

19. The cell population of claim 17, wherein the iGPSCs are human cells.

20. A method of stimulating growth of germline pluripotent stem cells (gPSCs), the method comprising
a) culturing the cells in the presence of a cell culture medium, and
b) inhibiting the activity of Rho kinase (ROCK) during culturing,
whereby culturing the gPSCs while inhibiting the activity of the Rho kinase will stimulate the growth of the gPSCs.

21. The method of claim 20, wherein the gPSCs are human cells.

22. The method of claim 20, wherein the gPSCs are not primary cells.

23. The method of claim 20, wherein the gPSCs are derived from spermatogonial stem cells or ovarian stem cells.

24. The method of claim 20, wherein the cell culture medium comprises serum or a serum replacement.

25. The method of claim 24, wherein the serum is human serum.

26. The method of claim 20, wherein the ROCK is Rho kinase inhibitor 1 (ROCK 1), Rho kinase inhibitor 2 (ROCK 2) or both.

27. The method of claim 20, wherein inhibiting the activity of ROCK comprises culturing the gPSCs in the presence of a small molecule ROCK inhibitor.

28. The method of claim 27, wherein the small molecule ROCK inhibitor is selected from the group consisting of Y-27632, HA1100 hydrochloride, HA1077 and GSK429286.

29. The method of claim 20, wherein inhibiting the activity of ROCK comprises culturing the gPSCs in the presence of an RNA interference (RNAi) molecule specific for ROCK 1, ROCK 2 or both.
